(19) United States
(12) Patent Application Publication
(10) Pub. No.: US 2014/0296316 A1
(43) Pub. Date: Oct. 2, 2014

(54) HYDRATE AND CRYSTAL OF FLUORENE COMPOUNDS

(71) Applicant: JAPAN TOBACCO INC., Tokyo (JP)

(72) Inventor: Takahisa Motomura, Takatsuki (JP)

(73) Assignee: JAPAN TOBACCO INC., Tokyo (JP)

(21) Appl. No.: 14/210,764

(22) Filed: Mar. 14, 2014

Related U.S. Application Data
(60) Provisional application No. 61/791,266, filed on Mar. 15, 2013.

(30) Foreign Application Priority Data
Mar. 15, 2013 (JP) 2013-053196

(51) Int. Cl. C07D 231/12

(52) U.S. Cl. CPC C07D 231/12; C07B 2200/13
USPC 514/406; 548/376.1

(57) ABSTRACT

A compound represented by the formula (J):

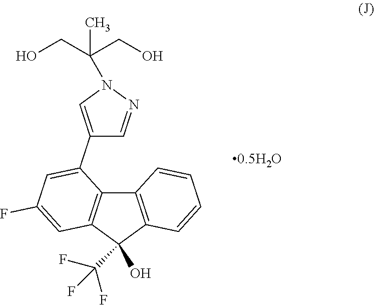 [SMILES: CC(CO)(CO)n1cc(-c2cc(F)cc3c2-c2ccccc2[C@]3(O)C(F)(F)F)cn1]

or a crystal thereof, or
a compound represented by the formula (Q):

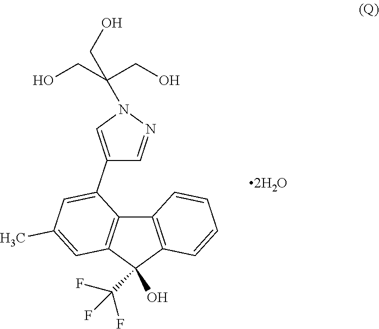 [SMILES: Cc1cc(-c2cnn(C(CO)(CO)CO)c2)c2c(c1)[C@@](O)(C(F)(F)F)c1ccccc1-2]

or a crystal thereof.

### TECHNICAL FIELD OF THE INVENTION

The present invention relates to a hydrate of a fluorene compound, and a crystal thereof. More particularly, the present invention relates to a hydrate of a fluorene compound having a pyruvate dehydrogenase kinase (PDHK) inhibitory action and properties superior in the stability, which is useful as a prophylactic or therapeutic agent for diabetes (type 1 diabetes, type 2 diabetes etc.), insulin resistance syndrome, metabolic syndrome, hyperglycemia, hyperlactacidemia, diabetic complications (diabetic neuropathy, diabetic retinopathy, diabetic nephropathy, cataract etc.), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension or Alzheimer disease, and a crystal thereof.

### BACKGROUND OF THE INVENTION

In tissues, for reactions using energy such as biosynthesis, active transport, muscle contraction and the like, the energy is supplied by hydrolysis of adenosine triphosphate (ATP). ATP is produced by oxidation of metabolic fuel which yields much energy, such as glucose and free fatty acids. In oxidative tissues such as muscle, ATP is mostly produced from acetyl-CoA that enters citric acid cycle. Acetyl-CoA is produced by oxidation of glucose via glycolytic pathway or β oxidation of free fatty acid. An enzyme that plays a pivotal role in controlling acetyl-CoA production from glucose is pyruvate dehydrogenase (hereinafter to be abbreviated as PDH). PDH catalyzes reduction of nicotinamide adenine dinucleotide (NAD) to NADH, simultaneously with oxidation of pyruvic acid to acetyl-CoA and carbon dioxide (e.g., non-patent documents 1, 2).

PDH is a multienzyme complex consisting of three enzyme components (E1, E2 and E3) and some subunits localized in mitochondrial matrix. E1, E2 and E3 are responsible for decarboxylation from pyruvic acid, production of acetyl-CoA and reduction of NAD to NADH, respectively.

Two classes of enzyme having regulatory function bind to PDH. One is PDHK, which is a protein kinase having specificity PDH. The role thereof is to inactivate E1α subunit of the complex by phosphorylation. The other is PDH phosphatase, which is a specific protein phosphatase that activates PDH via dephosphorylation of E1α subunit. The proportion of PDH in its active (dephosphorylated) state is determined by the balance of kinase activity and phosphatase activity. The kinase activity is regulated by the relative concentration of metabolic substrates. For example, the kinase activity is activated by an increase in NADH/NAD, acetyl-CoA/CoA and ATP/adenosine diphosphate (ADP) ratios, and inhibited by pyruvic acid (e.g., non-patent document 3).

In the tissues of mammals, 4 kinds of PDHK isozymes are identified. Particularly, PDHK2 is expressed in a wide range of tissues including the liver, skeletal muscles and adipose tissues involved in glucose metabolism. Furthermore, since PDHK2 shows comparatively high sensitivity to activation by increased NADH/NAD or acetyl-CoA/CoA and inhibition by pyruvic acid, involvement in a short-term regulation of glucose metabolism is suggested (e.g., non-patent document 4).

In addition, PDHK1 is expressed in large amounts in cardiac muscle, skeletal muscle, pancreatic β cell and the like. Furthermore, since expression of PDHK1 is induced via activation of hypoxia inducible factor (HIF) 1 in ischemic state, its involvement in ischemic diseases and cancerous diseases is suggested (e.g., non-patent document 5).

In diseases such as insulin-dependent (type 1) diabetes, non-insulin-dependent (type 2) diabetes and the like, oxidation of lipids is promoted with simultaneous reduction in glucose utilization. This reduction in glucose utilization is one of the factors causing hyperglycemia. When the oxidative glucose metabolism decreases in type 1 and type 2 diabetes and obesity, PDH activity also decreases. It suggests involvement of reduced PDH activity in the reduced glucose utilization in type 1 and type 2 diabetes (e.g., non-patent documents 6, 7).

On the contrary, hepatic gluconeogenesis is enhanced in type 1 and type 2 diabetes, which also forms one factor causing hyperglycemia. The reduced PDH activity increases pyruvic acid concentration, which in turn increases availability of lactic acid as a substrate for hepatic gluconeogenesis. It suggests possible involvement of reduced PDH activity in the enhanced gluconeogenesis in type 1 and type 2 diabetes (e.g., non-patent documents 8, 9). When PDH is activated by inhibition of PDHK, the rate of glucose oxidation is considered to rise. As a result, glucose utilization in the body is promoted and hepatic gluconeogenesis is suppressed, whereby hyperglycemia in type 1 and type 2 diabetes is expected to be improved (e.g., non-patent documents 10, 11, 12). Another factor contributing to diabetes is impaired insulin secretion, which is known to be associated with reduced PDH activity in pancreatic β cells, and introduction of PDHK1, 2 and 4 (e.g., non-patent documents 13, 14). In addition, sustained hyperglycemia due to diabetes is known to cause complications such as diabetic neuropathy, diabetic retinopathy, diabetic nephropathy and the like. Thiamine and α-lipoic acid contribute to activation of PDH as coenzymes. Thiamine and α-lipoic acid, or thiamine derivative and α-lipoic acid derivative are shown to have a promising effect on the treatment of diabetic complications. Thus, activation of PDH is expected to improve diabetic complications (e.g., non-patent documents 15, 16).

Under ischemic conditions, limited oxygen supply reduces oxidation of both glucose and fatty acid and reduces the amount of ATP produced by oxidative phosphorylation in the tissues. In the absence of sufficient oxygen, ATP level is maintained by promoted anaerobic glycolysis. As a result, lactic acid increases and intracellular pH decreases. Even though the body tries to maintain homeostasis of ion by energy consumption, abnormally low ATP level and disrupted cellular osmolarity lead to cell death. In addition, adenosine monophosphate-activating kinase, activated during ischemia, phosphorylates and thus inactivates acetyl-CoA carboxylase. The levels of total malonyl-CoA in the tissue drop, carnitine palmitoyltransferase-I activity is therefore increased and fatty acid oxidation is favored over glucose oxidation by allowing the transport of acyl-CoA into mitochondria. Oxidation of glucose is capable of yielding more ATP per molecule of oxygen than is oxidation of fatty acids. Under ischemic conditions, therefore, when energy metabolism becomes glucose oxidation dominant by activation of PDH, the ability to maintain ATP level is considered to be enhanced (e.g., non-patent document 17).

In addition, since activation of PDH causes oxidation of pyruvic acid produced by glycolysis, and reducing production of lactic acid, the net proton burden is considered to be reduced in ischemic tissues. Accordingly, PDH activation by inhibition of PDHK is expected to protectively act in ischemic diseases such as cardiac muscle ischemia (e.g., non-patent documents 18, 19).

A drug that activates PDH by inhibition of PDHK is considered to decrease lactate production since it promotes pyruvate metabolism. Hence, such drug is expected to be useful for the treatment of hyperlactacidemia such as mitochondrial disease, mitochondrial encephalomyopathy and sepsis (e.g., non-patent document 20).

In cancer cells, the expression of PDHK1 or 2 increases. In cancer cells, moreover, ATP production by oxidative phosphorylation in mitochondria decreases, and ATP production via the anaerobic glycolysis in cytoplasm increases. PDH activation by inhibition of PDHK is expected to promote oxidative phosphorylation in mitochondria, and increase production of active oxygen, which will induce apoptosis of cancer cells. Therefore, the PDH activation by PDHK inhibition is useful for the treatment of cancerous diseases (e.g., non-patent document 21).

Pulmonary hypertension is characterized by high blood pressure caused by partial narrowing of the pulmonary artery due to promoted cell proliferation therein. In pulmonary hypertension, therefore, activation of PDH in the pulmonary artery cell is expected to promote oxidative phosphorylation in mitochondria, increase production of active oxygen, and induce apoptosis of the pulmonary artery cells. Therefore, the PDH activation by PDHK inhibition is considered to be useful for the treatment of pulmonary hypertension (e.g., non-patent document 22).

Energy production and glucose metabolism in the cerebrum decrease in Alzheimer disease, and also, PDH activity declines. When the PDH activity declines, production of acetyl CoA decreases. Acetyl CoA is utilized for ATP production in the electron transport system via the citric acid cycle. Acetyl CoA is also a starting material for synthesizing acetylcholine, which is one of the neurotransmitters. Therefore, reduced brain PDH activity in Alzheimer disease is considered to cause neuronal cell death due to the decreased ATP production. Moreover, it is considered that synthesis of acetylcholine, which is the transmitter for cholinergic nerve, is inhibited to induce deterioration of memory and the like. Activation of PDH in the brain is expected to enhance energy production and acetylcholine synthesis in Alzheimer disease. Therefore, activation of PDH by the inhibition of PDHK is considered to be useful for the treatment of Alzheimer disease (e.g., non-patent documents 23, 24).

It has been shown that dichloroacetic acid, which is a drug having a PDH activating action, provides promising effects for the treatment of diabetes, myocardial ischemia, myocardial infarction, angina pectoris, cardiac failure, hyperlactacidemia, brain ischemia, cerebral apoplexy, peripheral arterial disease, chronic obstructive pulmonary disease, cancerous disease, and pulmonary hypertension (e.g., non-patent documents 10, 18, 20, 22, 25, 26, 27).

From the foregoing findings, a PDHK inhibitor is considered to be useful for the prophylaxis or treatment of diseases relating to glucose utilization disorder, for example, diabetes (type 1 diabetes, type 2 diabetes etc.), insulin resistance syndrome, metabolic syndrome, hyperglycemia, hyperlactacidemia, diabetic complications (diabetic neuropathy, diabetic retinopathy, diabetic nephropathy, cataract etc.). Furthermore, a PDHK inhibitor is considered to be useful for the prophylaxis or treatment of diseases caused by limited energy substrate supply to the tissues, for example, cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia and cerebral apoplexy.

Therefore, a PDHK inhibitor is considered to be useful for the prophylaxis or treatment of diabetes (type 1 diabetes, type 2 diabetes etc.), insulin resistance syndrome, metabolic syndrome, hyperglycemia, hyperlactacidemia, diabetic complications (diabetic neuropathy, diabetic retinopathy, diabetic nephropathy, cataract etc.), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension or Alzheimer disease.

### DOCUMENT LIST

### Non-Patent Documents

### Non-Patent Document 1:

Reed L J, Hackert M L. Structure-function relationships in dihydrolipoamide acyltransferases. J Biol Chem. 1990 Jun. 5; 265(16): 8971-4.

### Non-Patent Document 2:

Patel M S, Roche T E. Molecular biology and biochemistry of pyruvate dehydrogenase complexes. FASEB J. 1990 November; 4(14): 3224-33.

### Non-Patent Document 3:

Sugden M C, Holness M J. Recent advances in mechanisms regulating glucose oxidation at the level of the pyruvate dehydrogenase complex by PDKs. Am J Physiol Endocrinol Metab. 2003 May; 284(5): 5855-62.

### Non-Patent Document 4:

Bowker-Kinley M M, Davis W I, Wu P, Harris R A, Popov K M. Evidence for existence of tissue-specific regulation of the mammalian pyruvate dehydrogenase complex. Biochem J. 1998 Jan. 1; 329 (Pt 1): 191-6.

### Non-Patent Document 5:

Kim J W, Tchernyshyov I, Semenza G L, Dang C V. HIF-1-mediated expression of pyruvate dehydrogenase kinase: a metabolic switch required for cellular adaptation to hypoxia. Cell Metab. 2006 March; 3(3): 177-85.

### Non-Patent Document 6:

Morino K, Petersen K F, Dufour S, Befroy D, Frattini J, Shatzkes N, et al. Reduced mitochondrial density and increased IRS-1 serine phosphorylation in muscle of insulin-resistant offspring of type 2 diabetic parents. J Clin Invest. 2005 December; 115(12): 3587-93.

### Non-Patent Document 7:

Caterson I D, Fuller S J, Randle P J. Effect of the fatty acid oxidation inhibitor 2-tetradecylglycidic acid on pyruvate dehydrogenase complex activity in starved and alloxan-diabetic rats. Biochem J. 1982 Oct. 15; 208(1): 53-60.

### Non-Patent Document 8:

Boden G, Chen X, Stein T P. Gluconeogenesis in moderately and severely hyperglycemic patients with type 2 diabetes mellitus. Am J Physiol Endocrinol Metab. 2001 January; 280(1): E23-30.

### Non-Patent Document 9:

Shangraw R E, Fisher D M. Pharmacokinetics and pharmacodynamics of dichloroacetate in patients with cirrhosis. Clin Pharmacol Ther. 1999 October; 66(4): 380-90.

### Non-Patent Document 10:

Stacpoole P W, Moore G W, Kornhauser D M. Metabolic effects of dichloroacetate in patients with diabetes mellitus and hyperlipoproteinemia. N Engl J Med. 1978 Mar. 9; 298(10): 526-30.

### Non-Patent Document 11:

Mayers R M, Leighton B, Kilgour E. PDH kinase inhibitors: a novel therapy for Type II diabetes? Biochem Soc Trans. 2005 April; 33(Pt 2): 367-70.

### Non-Patent Document 12:

Jeoung N H, Rahimi Y, Wu P, Lee W N, Harris R A. Fasting induces ketoacidosis and hypothermia in PDHK2/PDHK4-double-knockout mice. Biochem J. 2012 May 1; 443(3): 829-39.

### Non-Patent Document 13:

Zhou Y P, Berggren P O, Grill V. A fatty acid-induced decrease in pyruvate dehydrogenase activity is an important determinant of beta-cell dysfunction in the obese diabetic db/db mouse. Diabetes. 1996 May; 45(5): 580-6.

### Non-Patent Document 14:

Xu J, Han J, Epstein P N, Liu Y Q. Regulation of PDK mRNA by high fatty acid and glucose in pancreatic islets. Biochem Biophys Res Commun. 2006 Jun. 9; 344(3): 827-33.

### Non-Patent Document 15:

Benfotiamine. Monograph. Altern Med Rev. 2006 September; 11(3): 238-42.

### Non-Patent Document 16:

Vallianou N, Evangelopoulos A, Koutalas P. Alpha-lipoic Acid and diabetic neuropathy. Rev Diabet Stud. 2009 Winter; 6(4): 230-6.

### Non-Patent Document 17:

Ussher J R, Lopaschuk G D. The malonyl CoA axis as a potential target for treating ischaemic heart disease. Cardiovasc Res. 2008 Jul. 15; 79(2): 259-68.

### Non-Patent Document 18:

Wargovich T J, MacDonald R G, Hill J A, Feldman R L, Stacpoole P W, Pepine C J. Myocardial metabolic and hemodynamic effects of dichloroacetate in coronary artery disease. Am J Cardiol. 1988 Jan. 1; 61(1): 65-70.

### Non-Patent Document 19:

Taniguchi M, Wilson C, Hunter C A, Pehowich D J, Clanachan A S, Lopaschuk G D. Dichloroacetate improves cardiac efficiency after ischemia independent of changes in mitochondrial proton leak. Am J Physiol Heart Circ Physiol. 2001 April; 280(4): H1762-9.

### Non-Patent Document 20:

Stacpoole P W, Nagaraja N V, Hutson A D. Efficacy of dichloroacetate as a lactate-lowering drug. J Clin Pharmacol. 2003 July; 43(7): 683-91.

### [Non-Patent Document 21]

Bonnet S, Archer S L, Allalunis-Turner J, Haromy A, Beaulieu C, Thompson R, et al. A mitochondria-K+ channel axis is suppressed in cancer and its normalization promotes apoptosis and inhibits cancer growth. Cancer Cell. 2007 January; 11(1): 37-51.

### [Non-Patent Document 22]

McMurtry M S, Bonnet S, Wu X, Dyck J R, Haromy A, Hashimoto K, et al. Dichloroacetate prevents and reverses pulmonary hypertension by inducing pulmonary artery smooth muscle cell apoptosis. Circ Res. 2004 Oct. 15; 95(8): 830-40.

### [Non-Patent Document 23]

Saxena U. Bioenergetics breakdown in Alzheimer's disease: targets for new therapies. Int J Physiol Pathophysiol Pharmacol. 2011; 3(2): 133-9.

### [Non-Patent Document 24]

Stacpoole P W. The pyruvate dehydrogenase complex as a therapeutic target for age-related diseases. Aging Cell. 2012 June; 11(3): 371-7.

### [Non-Patent Document 25]

Marangos P J, Turkel C C, Dziewanowska Z E, Fox A W. Dichloroacetate and cerebral ischaemia therapeutics. Expert Opin Investig Drugs. 1999 April; 8(4): 373-82.

### [Non-Patent Document 26]

Calvert L D, Shelley R, Singh S J, Greenhaff P L, Bankart J, Morgan M D, et al. Dichloroacetate enhances performance and reduces blood lactate during maximal cycle exercise in chronic obstructive pulmonary disease. Am J Respir Crit Care Med. 2008 May 15; 177(10): 1090-4.

### [Non-Patent Document 27]

Flavin D F. Non-Hodgkin's Lymphoma Reversal with Dichloroacetate. J Oncol. Hindawi Publishing Corporation Journal of Oncology Volume 2010, Article ID 414726, 4 pages doi:10.1155/2010/414726.

### SUMMARY OF THE INVENTION

The present invention is as follow.

[1] A compound represented by the formula (J):

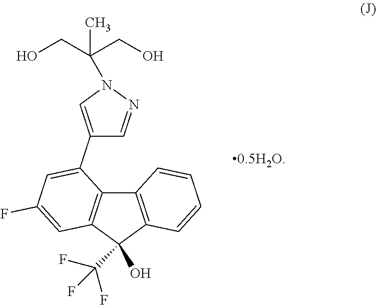 [SMILES: CC(CO)(CO)n1cc(-c2cc(F)cc3c2-c2ccccc2[C@]3(O)C(F)(F)F)cn1]

[2] A crystal of the compound of the above-mentioned [1].
[3] A crystal of the compound of the above-mentioned [1], having peaks at diffraction angles 2θ(°) of 6.9±0.2, 10.2±0.2, 15.5±0.2, 15.8±0.2 and 16.6±0.2 in powder X-ray diffraction.
[4] A crystal of the compound of the above-mentioned [1], having peaks at diffraction angles 2θ(°) of about 6.9, about 10.2, about 15.5, about 15.8 and about 16.6 in powder X-ray diffraction.
[5] A crystal of the compound of the above-mentioned [1], having peaks at diffraction angles 2θ(°) of 6.9, 10.2, 15.5, 15.8 and 16.6 in powder X-ray diffraction.
[6] A crystal of the compound of the above-mentioned [1], having a peak at diffraction angle 2θ(°) of 10.2±0.2 in powder X-ray diffraction.
[7] A crystal of the compound of the above-mentioned [1], having the following peaks in powder X-ray diffraction spectrum:

TABLE 1

| | relative intensity | NET intensity |
|---|---|---|
| Pos. [°2Th.] | [%] | [cts] |
| 3.3317 | 2.77 | 200.57 |
| 6.8793 | 74.26 | 5386.14 |
| 8.2956 | 1.98 | 143.93 |
| 10.1647 | 100.00 | 7252.64 |
| 10.6533 | 6.35 | 460.64 |
| 11.4229 | 14.04 | 1018.53 |
| 12.6712 | 1.36 | 98.28 |
| 13.0816 | 1.96 | 141.80 |
| 13.5502 | 44.85 | 3252.99 |
| 13.7982 | 18.62 | 1350.56 |
| 13.9984 | 19.91 | 1444.07 |
| 15.5328 | 56.99 | 4133.20 |
| 15.7650 | 65.82 | 4773.94 |
| 16.6441 | 68.86 | 4994.32 |
| 17.1335 | 19.33 | 1401.80 |
| 17.4440 | 6.44 | 466.71 |
| 18.1837 | 17.95 | 1301.61 |
| 18.5774 | 47.73 | 3461.98 |
| 18.7787 | 32.67 | 2369.39 |
| 20.3793 | 10.30 | 746.72 |
| 20.7151 | 33.59 | 2436.49 |
| 21.4580 | 9.06 | 657.01 |
| 21.7939 | 3.43 | 248.99 |
| 22.1436 | 46.75 | 3390.28 |
| 22.6122 | 18.48 | 1339.95 |
| 22.9773 | 11.30 | 819.87 |
| 23.3168 | 4.15 | 301.26 |
| 23.8856 | 5.45 | 395.23 |
| 24.1980 | 5.71 | 414.43 |
| 24.4588 | 3.52 | 255.36 |

[8] The crystal of any of the above-mentioned [2] to [7], having an extrapolated onset temperature of 88.7±5.0° C. in differential scanning calorimetry.
[9] The compound of the above-mentioned [1], which is a crystal of any of the above-mentioned [3] to [8] having a purity of not less than 70%.
[10] A pharmaceutical composition comprising the compound of any of the above-mentioned [1] to [9] or a crystal thereof, and a pharmaceutically acceptable carrier.
[11] The pharmaceutical composition of the above-mentioned [10], which is a granule, a fine granule, a powder, a capsule or a tablet.
[12] A PDHK inhibitor comprising the compound of any of the above-mentioned [1] to [9] or a crystal thereof, and a pharmaceutically acceptable carrier.
[13] A PDHK1 inhibitor comprising the compound of any of the above-mentioned [1] to [9] or a crystal thereof, and a pharmaceutically acceptable carrier.
[14] A PDHK2 inhibitor comprising the compound of any of the above-mentioned [1] to [9] or a crystal thereof, and a pharmaceutically acceptable carrier.
[15] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension, comprising the compound of any of the above-mentioned [1] to [9] or a crystal thereof, and a pharmaceutically acceptable carrier.
[15′] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease, comprising the compound of any of the above-mentioned [1] to [9] or a crystal thereof, and a pharmaceutically acceptable carrier.
[16] A method of inhibiting PDHK in a mammal, comprising administering a pharmaceutically effective amount of the compound of any of the above-mentioned [1] to [9] or a crystal thereof to the mammal.
[17] A method of preventing or treating a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension in a mammal, comprising administering a pharmaceutically effective amount of the compound of any of the above-mentioned [1] to [9] or a crystal thereof to the mammal.
[18] Use of the compound of any of the above-mentioned [1] to [9] or a crystal thereof for the production of a PDHK inhibitor.
[19] Use of the compound of any of the above-mentioned [1] to [9] or a crystal thereof for the production of a prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[20] Use of the above-mentioned [18] or [19] in combination with at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[21] A pharmaceutical composition comprising
(a) the compound of any of the above-mentioned [1] to [9] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[21′] A pharmaceutical composition comprising
(a) the compound of any of the above-mentioned [1] to [9] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease.
[22] A combination drug comprising
(a) the compound of any of the above-mentioned [1] to [9] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer and pulmonary hypertension, which are administered simultaneously, separately or continuously.
[22′] A combination drug comprising
(a) the compound of any of the above-mentioned [1] to [9] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension and Alzheimer disease, which are administered simultaneously, separately or continuously.
[23] A crystal of the compound of the above-mentioned [1], having peaks at diffraction angles 2θ(°) of 6.9±0.2, 10.2±0.2, 13.6±0.2, 15.8±0.2 and 16.6±0.2 in powder X-ray diffraction.
[24] A crystal of the compound of the above-mentioned [1], having peaks at diffraction angles 2θ(°) of about 6.9, about 10.2, about 13.6, about 15.8 and about 16.6 in powder X-ray diffraction.
[25] A crystal of the compound of the above-mentioned [1], having peaks at diffraction angles 2θ(°) of 6.9, 10.2, 13.6, 15.8 and 16.6 in powder X-ray diffraction.
[26] The compound of the above-mentioned [1], which is a crystal of any of the above-mentioned [23] to [25] having a purity of not less than 70%.
[27] A pharmaceutical composition comprising the compound of any of the above-mentioned [23] to [26] or a crystal thereof, and a pharmaceutically acceptable carrier.
[28] The pharmaceutical composition of the above-mentioned [27], which is a granule, a fine granule, a powder, a capsule or a tablet.
[29] A PDHK inhibitor comprising the compound of any of the above-mentioned [23] to [26] or a crystal thereof, and a pharmaceutically acceptable carrier.
[30] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension, comprising the compound of any of the above-mentioned [23] to [26] or a crystal thereof, and a pharmaceutically acceptable carrier.
[30′] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease, comprising the compound of any of the above-mentioned [23] to [26] or a crystal thereof, and a pharmaceutically acceptable carrier.
[31] A method of inhibiting PDHK in a mammal, comprising administering a pharmaceutically effective amount of the compound of any of the above-mentioned [23] to [26] or a crystal thereof to the mammal.
[32] A method of preventing or treating a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension in a mammal, comprising administering a pharmaceutically effective amount of the compound of any of the above-mentioned [23] to [26] or a crystal thereof to the mammal.
[33] Use of the compound of any of the above-mentioned [23] to [26] or a crystal thereof for the production of a PDHK inhibitor.
[34] Use of the compound of any of the above-mentioned [23] to [26] or a crystal thereof for the production of a prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[35] Use of the above-mentioned [18] or [19] in combination with at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[36] A pharmaceutical composition comprising
(a) the compound of any of the above-mentioned [23] to [26] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[36′] A pharmaceutical composition comprising
(a) the compound of any of the above-mentioned [23] to [26] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease.
[37] A combination drug comprising
(a) the compound of any of the above-mentioned [23] to [26] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer and pulmonary hypertension, which are administered simultaneously, separately or continuously.
[37′] A combination drug comprising
(a) the compound of any of the above-mentioned [23] to [26] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension and Alzheimer disease, which are administered simultaneously, separately or continuously.
[38] A crystal form mixture comprising the crystal of any of the above-mentioned [3] to [8] and the crystal of any of the above-mentioned [23] to [25].
[39] The crystal form mixture of the above-mentioned [38], wherein the crystal of any of the above-mentioned [3] to [8] is contained in not less than 70%.
[40] The crystal form mixture of the above-mentioned [38], wherein the crystal of any of the above-mentioned [23] to [25] is contained in not less than 70%.
[41] A pharmaceutical composition comprising the crystal form mixture of any of the above-mentioned [38] to [40].
[42] The pharmaceutical composition of the above-mentioned [41], which is a granule, a fine granule, a powder, a capsule or a tablet.
[43] A production method of a crystal of 0.5 hydrate of 2-{4-[(9R)-2-fluoro-9-hydroxy-9-(trifluoromethyl)-9H-fluoren-4-yl]-pyrazol-1-yl}-2-methyl-propane-1,3-diol, comprising
(a) mixing 2-{4-[(9R)-2-fluoro-9-hydroxy-9-(trifluoromethyl)-9H-fluoren-4-yl]-pyrazol-1-yl}-2-methyl-propane-1,3-diol with water or a water-containing solvent,
(b) stirring and/or standing the mixture until 0.5 hydrate of 2-{4-[(9R)-2-fluoro-9-hydroxy-9-(trifluoromethyl)-9H-fluoren-4-yl]-pyrazol-1-yl}-2-methyl-propane-1,3-diol is formed, and
(c) stirring and/or standing the mixture until the crystal of any of the above-mentioned [3] to [8] or [23] to [25] is precipitated.
[44] A compound represented by the formula (Q):

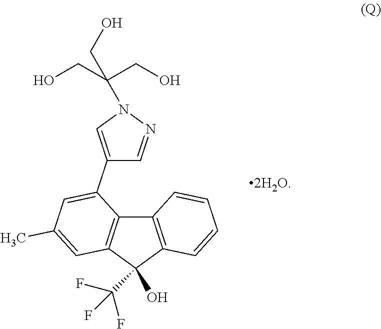 [SMILES: Cc1cc(-c2cnn(C(CO)(CO)CO)c2)c2c(c1)[C@@](O)(C(F)(F)F)c1ccccc1-2]

[45] A crystal of the compound of the above-mentioned [44].
[46] A crystal of the compound of the above-mentioned [44], having peaks at diffraction angles 2θ(°) of 11.8±0.2, 13.2±0.2, 14.3±0.2, 16.6±0.2 and 19.8±0.2 in powder X-ray diffraction.
[47] A crystal of the compound of the above-mentioned [44], having peaks at diffraction angles 2θ(°) of about 11.8, about 13.2, about 14.3, about 16.6 and about 19.8 in powder X-ray diffraction.
[48] A crystal of the compound of the above-mentioned [44], having peaks at diffraction angles 2θ(°) of 11.8, 13.2, 14.3, 16.6 and 19.8 in powder X-ray diffraction.
[49] The crystal of any of the above-mentioned [45] to [48], having an extrapolated onset temperature of 62.3±5.0° C. in differential scanning calorimetry.
[50] A crystal of the compound of the above-mentioned [44], having the following peaks in powder X-ray diffraction spectrum:

TABLE 2

| | relative | NET |
|---|---|---|
| | intensity | intensity |
| Pos. [°2Th.] | [%] | [cts] |
| 6.5961 | 15.20 | 962.89 |
| 10.2647 | 13.13 | 831.67 |
| 11.7561 | 61.24 | 3878.21 |
| 13.2157 | 61.19 | 3875.41 |
| 13.8990 | 56.48 | 3576.72 |
| 14.2972 | 76.28 | 4830.78 |
| 15.4027 | 18.18 | 1151.63 |
| 16.5556 | 100.00 | 6333.25 |
| 17.1010 | 20.99 | 1329.07 |
| 19.2254 | 25.81 | 1634.70 |
| 19.8022 | 69.77 | 4418.89 |
| 20.1950 | 3.38 | 214.11 |
| 20.6126 | 4.13 | 261.78 |
| 20.9929 | 2.67 | 169.00 |
| 21.6665 | 55.90 | 3540.60 |
| 21.9230 | 35.28 | 2234.41 |
| 22.1797 | 56.08 | 3551.80 |
| 23.1860 | 9.82 | 621.92 |
| 23.9516 | 23.38 | 1480.48 |
| 24.1124 | 32.65 | 2067.75 |
| 24.3616 | 13.51 | 855.65 |

[51] The compound of the above-mentioned [44], which is a crystal of any of the above-mentioned [46] to [50] having a purity of not less than 70%.
[52] A pharmaceutical composition comprising the compound of any of the above-mentioned [44] to [51] or a crystal thereof, and a pharmaceutically acceptable carrier.
[53] The pharmaceutical composition of the above-mentioned [52], which is a granule, a fine granule, a powder, a capsule or a tablet.
[54] A PDHK inhibitor comprising the compound of any of the above-mentioned [44] to [51] or a crystal thereof, and a pharmaceutically acceptable carrier.
[55] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension, comprising the compound of any of the above-mentioned [44] to [51] or a crystal thereof, and a pharmaceutically acceptable carrier.
[55′] A prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic so complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease, comprising the compound of any of the above-mentioned [44] to [51] or a crystal thereof, and a pharmaceutically acceptable carrier.
[56] A method of inhibiting PDHK in a mammal, comprising administering a pharmaceutically effective amount of the compound of any of the above-mentioned [44] to [51] or a crystal thereof to the mammal.
[57] A method of preventing or treating a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension in a mammal, comprising administering a pharmaceutically effective amount of the compound of any of the above-mentioned [44] to [51] or a crystal thereof to the mammal.
[58] Use of the compound of any of the above-mentioned [44] to [51] or a crystal thereof for the production of a PDHK inhibitor.
[59] Use of the compound of any of the above-mentioned [44] to [51] or a crystal thereof for the production of a prophylactic and/or therapeutic agent for a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[60] Use of the above-mentioned [58] or [59] in combination with at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[61] A pharmaceutical composition comprising
(a) the compound of any of the above-mentioned [44] to [51] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension.
[61′] A pharmaceutical composition comprising
(a) the compound of any of the above-mentioned [44] to [51] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer, pulmonary hypertension and Alzheimer disease.
[62] A combination drug comprising
(a) the compound of any of the above-mentioned [44] to [51] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer and pulmonary hypertension, which are administered simultaneously, separately or continuously.
[62′] A combination drug comprising
(a) the compound of any of the above-mentioned [44] to [51] or a crystal thereof, and
(b) at least one other medicament effective for the prophylaxis and/or treatment of a disease selected from the group consisting of diabetes (type 1 diabetes, type 2 diabetes), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension and Alzheimer disease, which are administered simultaneously, separately or continuously.
[63] A production method of a crystal of 2 hydrate of 2-hydroxymethyl-2-{4-[(9R)-9-hydroxy-2-methyl-9-(trifluoromethyl)-9H-fluoren-4-yl]-pyrazol-1-yl}-propane-1,3-diol, comprising
(a) mixing 2-hydroxymethyl-2-{4-[(9R)-9-hydroxy-2-methyl-9-(trifluoromethyl)-9H-fluoren-4-yl]-pyrazol-1-yl}-propane-1,3-diol with water or a water-containing solvent,
(b) stirring and/or standing the mixture until 2 hydrate of 2-hydroxymethyl-2-{4-[(9R)-9-hydroxy-2-methyl-9-(trifluoromethyl)-9H-fluoren-4-yl]-pyrazol-1-yl}-propane-1,3-diol is formed, and
(c) stirring and/or standing the mixture until the crystal of any of the above-mentioned [45] to [50] is precipitated.

### BRIEF DESCRIPTION OF THE DRAWINGS

FIG. 1 shows a powder X-ray diffraction pattern of the crystal of Example 1-1.

FIG. 2 shows a powder X-ray diffraction pattern of the crystal of Example 1-2.

FIG. 3 shows a powder X-ray diffraction pattern of the crystal of Example 1-3.

FIG. 4 shows a powder X-ray diffraction pattern of the crystal of Example 1-4.

FIG. 5 shows a powder X-ray diffraction pattern of the crystal of Example 1-5.

FIG. 6 shows a powder X-ray diffraction pattern of the crystal of Example 1-6.

FIG. 7 shows a powder X-ray diffraction pattern of the crystal of Example 1-7.

FIG. 8 shows a powder X-ray diffraction pattern of the crystal of Example 1-8.

FIG. 9 shows a powder X-ray diffraction pattern of the crystal of Example 1-9.

FIG. 10 shows a powder X-ray diffraction pattern of the crystal of Example 1-10.

FIG. 11 shows a powder X-ray diffraction pattern of the crystal of Example 2-1.

FIG. 12 shows a powder X-ray diffraction pattern of the crystal of Example 2-2.

FIG. 13 shows a powder X-ray diffraction pattern of the crystal of Example 2-3.

FIG. 14 shows a powder X-ray diffraction pattern of the crystal of Example 2-4.

FIG. 15 shows a powder X-ray diffraction pattern of the crystal of Example 2-5.

FIG. 16 shows a powder X-ray diffraction pattern of the crystal of Example 2-6.

FIG. 17 shows a powder X-ray diffraction pattern of the crystal of Example 2-7.

FIG. 18 shows a powder X-ray diffraction pattern of the crystal of Example 2-8.

FIG. 19 shows a powder X-ray diffraction pattern of the crystal of Example 2-9.

FIG. 20 shows a powder X-ray diffraction pattern of the crystal of Example 2-10.

FIG. 21 shows the DSC thermoanalytical data of the crystal of Example 1-1.

FIG. 22 shows the DSC thermoanalytical data of the crystal of Example 1-3.

FIG. 23 shows the DSC thermoanalytical data of the crystal of Example 2-1.

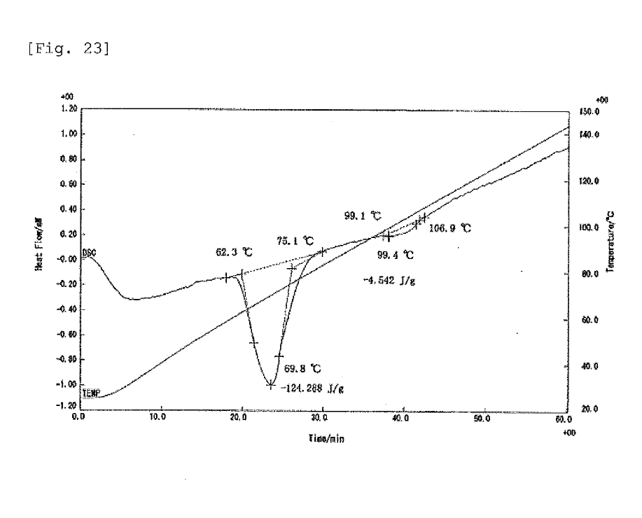

FIG. 24 shows the differential heat/thermogravimetry simultaneous measurement data of the crystal of Example 1-1.

FIG. 25 shows the differential heat/thermogravimetry simultaneous measurement data of the crystal of Example 1-2.

FIG. 26 shows the differential heat/thermogravimetry simultaneous measurement data of the crystal of Example 2-1.

FIG. 27 shows the water adsorption/desorption isothermal data of the crystal of Example 1-1 (without pre-drying).

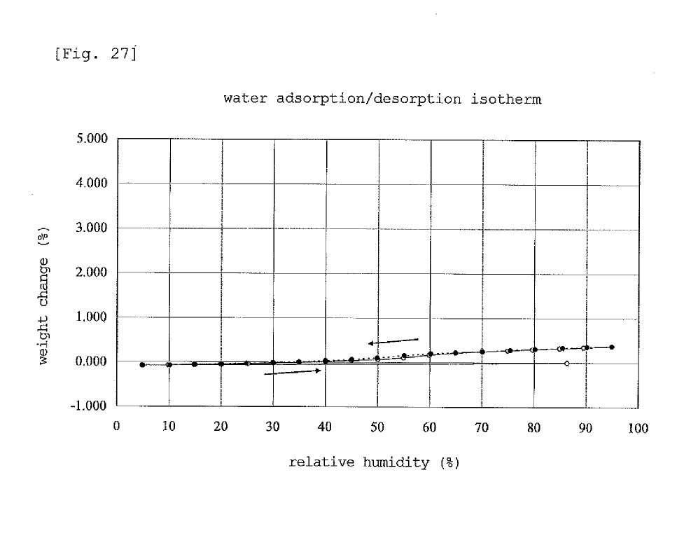

FIG. 28 shows the water adsorption/desorption isothermal data of the crystal of Example 1-1 (with pre-drying).

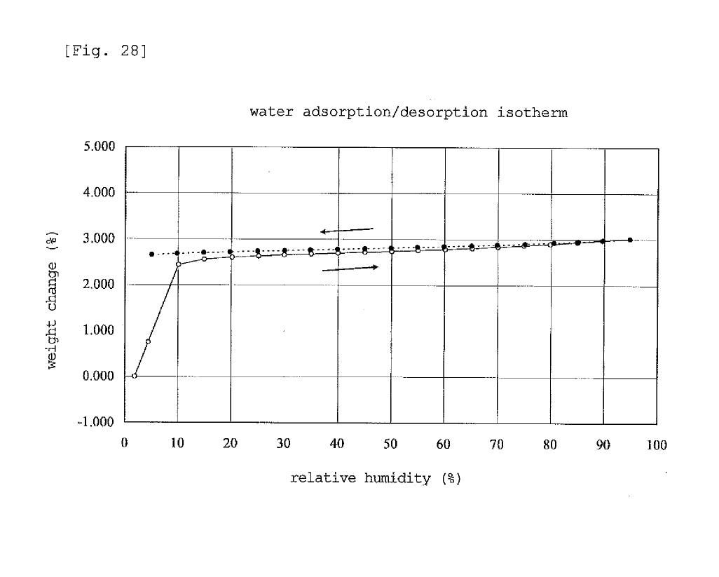

FIG. 29 shows the water adsorption/desorption isothermal data of the crystal of Example 1-2 (without pre-drying).

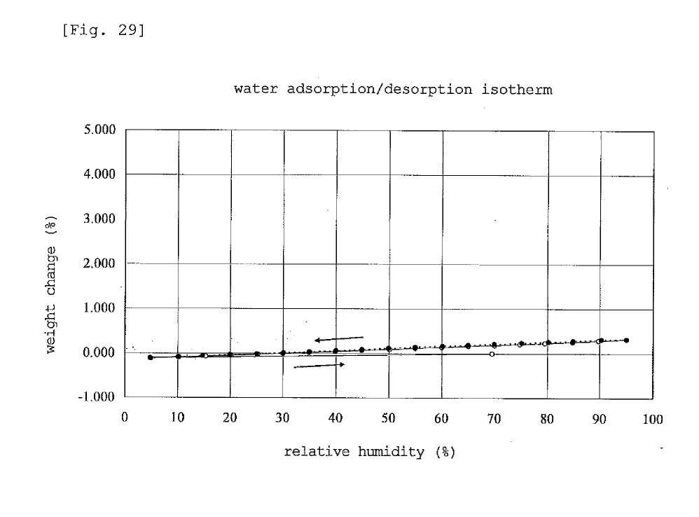

FIG. 30 shows the water adsorption/desorption isothermal data of the crystal of Example 1-2 (with pre-drying).

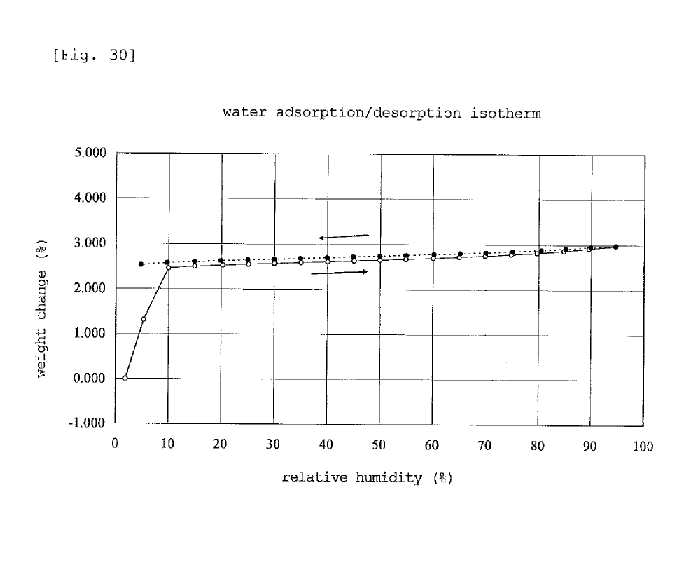

FIG. 31 shows the water adsorption/desorption isothermal data of the crystal of Example 2-1 (without pre-drying).

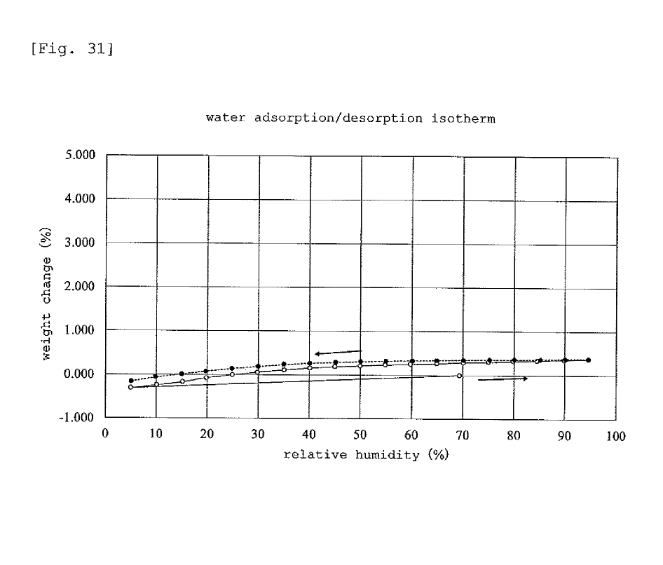

FIG. 32 shows the water adsorption/desorption isothermal data of the crystal of Example 2-1 (with pre-drying).

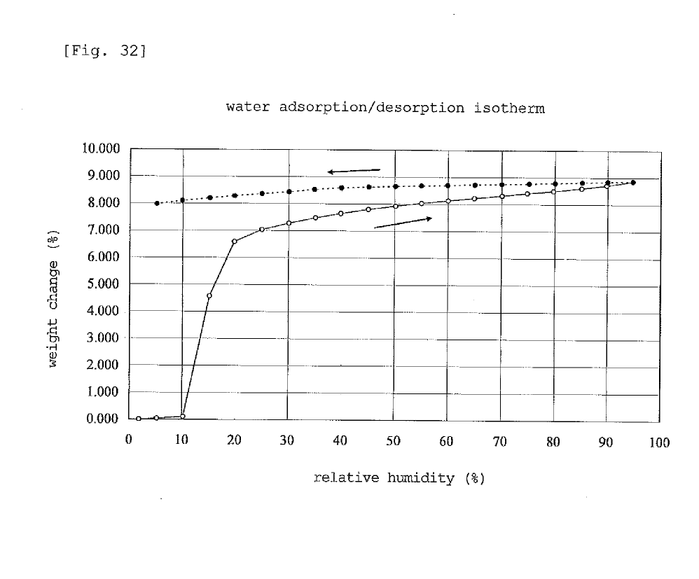

### DESCRIPTION OF EMBODIMENTS

The present invention is explained in detail in the following.

2-{4-[(9R)-2-Fluoro-9-hydroxy-9-(trifluoromethyl)-9H-fluoren-4-yl]-pyrazol-1-yl}-2-methyl-propane-1,3-diol (WO 2010/041748) is hereinafter sometimes to be referred to as “compound A”.

2-Hydroxymethyl-2-{4-[(9R)-9-hydroxy-2-methyl-9-(trifluoromethyl)-9H-fluoren-4-yl]-pyrazol-1-yl}-propane-1,3-diol (WO 2010/041748) is hereinafter sometimes to be referred to as “compound B”.

One aspect of the present invention is 0.5 hydrate of compound A (hereinafter sometimes to be referred to as compound (Ah)). One embodiment is shown by the following chemical formula.

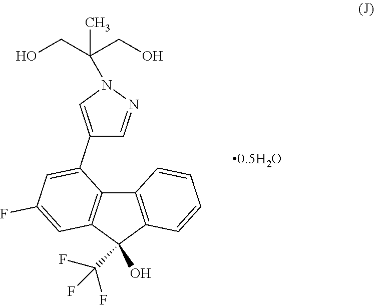 [SMILES: CC(CO)(CO)n1cc(-c2cc(F)cc3c2-c2ccccc2[C@]3(O)C(F)(F)F)cn1]

One embodiment is shown by the following chemical formula.

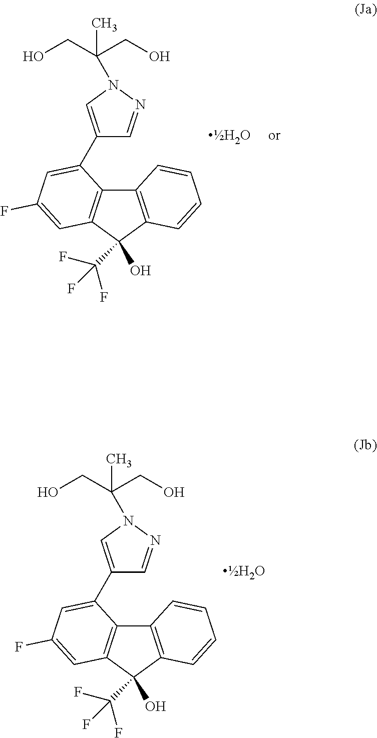 [SMILES: CC(CO)(CO)n1cc(-c2cc(F)cc3c2-c2ccccc2[C@]3(O)C(F)(F)F)cn1 ; CC(CO)(CO)n1cc(-c2cc(F)cc3c2-c2ccccc2[C@]3(O)C(F)(F)F)cn1]

Another embodiment is shown by the following chemical formula.

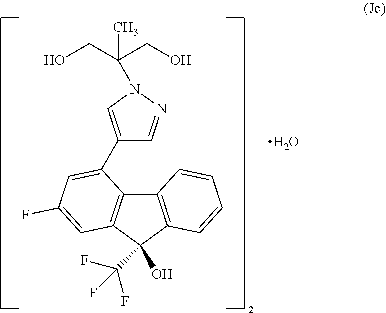

Moreover, one aspect of the present invention is 2 hydrate of compound B (hereinafter sometimes to be referred to as compound (Bh)). One embodiment is shown by the following chemical formula.

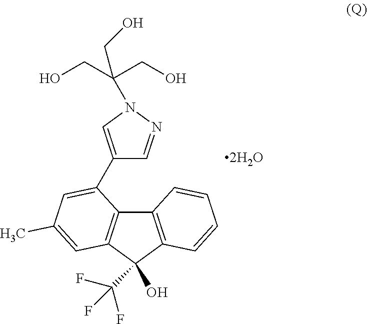 [SMILES: Cc1cc(-c2cnn(C(CO)(CO)CO)c2)c2c(c1)[C@@](O)(C(F)(F)F)c1ccccc1-2]

One of the crystals of the present invention can be produced by, for example, crystal transition of amorphous compound A (including solvate (hydrate etc.)), or other crystal of compound A (including solvate (hydrate etc.)). Moreover, one of the crystals of the present invention can be produced by, for example, crystal transition of amorphous compound B (including solvate (hydrate etc.)), or other crystal of compound B (including solvate (hydrate etc.)).

To obtain the crystal of compound (Ah) of the present invention, a slurry method using water or an aprotic solvent as a solvent is preferable from among the above-mentioned methods.

As an analytical method of the obtained crystal, a crystal analysis method based on X-ray diffraction is generally employed. Furthermore, as a method for confirming the crystal form, a mechanical method or an optical method (e.g., FT-Raman spectrum, solid-state NMR spectrum) and the like can also be mentioned. In addition, thermoanalysis (Differential Scanning Calorimetry (DSC)), infrared absorption spectroscopic analysis and the like of the crystal can also be performed according to a general method.

In the present invention, the “Form I crystal” of compound (Ah) means a crystal of compound (I), which shows an X-ray powder diffraction pattern having a characteristic peak at a diffraction angle 2θ(°) of 10.2°, as measured by powder X-ray diffraction.

One embodiment of the “Form I crystal” of compound (Ah) is a crystal of compound (Ah) showing an X-ray powder diffraction pattern having peaks at diffraction angles 2θ of

(1) 6.9, 10.2, 13.6, 13.8, 14.0, 15.5, 15.8, 16.6, 17.1, 18.2, 18.6, 18.8, 20.7, 22.1 and 22.6°,
(2) 6.9, 10.2, 13.6, 15.5, 15.8, 16.6, 18.6, 18.8, 20.7 and 22.1°,
(3) 6.9, 10.2, 15.5, 15.8, 16.6 and 18.6°, or
(4) 6.9, 10.2, 13.6, 13.8 and 14.0°, as measured by powder X-ray diffraction.

In the present invention, the “Form I crystal” of compound (Ah) is preferably a crystal of compound (Ah), which shows an X-ray powder diffraction pattern having characteristic peaks at diffraction angles 2θ(°) of 6.9, 10.2, 15.5, 15.8 and 16.6°, as measured by powder X-ray diffraction.

In the present invention, the “Form I crystal” of compound (Ah) is more preferably a crystal of compound (Ah), which shows an X-ray powder diffraction pattern having characteristic peaks at diffraction angles 2θ(°) of 6.9, 10.2, 13.6, 15.5, 15.8, 16.6, 18.6, 18.8, 20.7 and 22.1°, as measured by powder X-ray diffraction.

In the present invention, the “Form V crystal” of compound (Ah) means a crystal of compound (Ah), which shows an X-ray powder diffraction pattern having characteristic peaks at diffraction angles 2θ(°) of 6.9, 10.2, 13.6, 15.8 and 16.6°, as measured by powder X-ray diffraction.

In the present invention, the “Form V crystal” of compound (Ah) is more preferably a crystal of compound (Ah), which shows an X-ray powder diffraction pattern having characteristic peaks at diffraction angles 2θ(°) of 6.9, 10.2, 13.6, 15.5, 15.8, 16.6, 18.5, 20.6, 22.1 and 22.7°, as measured by powder X-ray diffraction.

In the present invention, the “Form IVb crystal” of compound (Bh) means a crystal of compound (Bh), which shows an X-ray powder diffraction pattern having characteristic peaks at diffraction angles 2θ(°) of 11.8, 13.2, 14.3, 16.6 and 19.8°, as measured by powder X-ray diffraction.

One embodiment of the “Form IVb crystal” of compound (Bh) is a crystal of compound (Bh) showing an X-ray powder diffraction pattern having peaks at diffraction angles 2θ of

(1) 6.6, 11.8, 13.2, 13.9, 14.3, 15.4, 16.6, 17.1, 19.2, 19.8, 21.7, 21.9, 22.2, 24.0 and 24.1°,
(2) 11.8, 13.2, 13.9, 14.3, 16.6, 19.8, 21.7, 21.9, 22.2 and 24.1°,
(3) 11.8, 13.2, 13.9, 14.3, 16.6 and 19.8°,
(4) 11.8, 13.2, 14.3, 16.6 and 19.8°, or
(5) 6.6, 11.8, 13.2, 13.9 and 14.3°, as measured by powder X-ray diffraction.

In the present invention, the “Form IVb crystal” of compound (Bh) is more preferably a crystal of compound (Bh), which shows an X-ray powder diffraction pattern having characteristic peaks at diffraction angles 2θ(°) of 11.8, 13.2, 13.9, 14.3, 16.6, 19.8, 21.7, 21.9, 22.2 and 24.1°, as measured by powder X-ray diffraction.

The diffraction peak value in the above-mentioned diffraction angle 2θ(°) sometimes shows a measurement error of some level due to the measurement device, measurement conditions and the like Specifically, the measurement error is within the range of ±0.2, preferably ±0.1, more preferably ±0.06. A diffraction peak value containing a measurement error is sometimes indicated with an “about”.

Compound (Ah), compound (Bh), and crystals thereof in the present invention (hereinafter these are sometimes to be generically abbreviated as “the compound of the present invention”) are also characterized by thermoanalysis. For example, when Form I crystal of compound (Ah) is subjected to DSC measurement, the enthalpy of the endothermic peak is about 79.2 J/g, and the extrapolated onset temperature is 88.7±5.0° C. When, for example, Form IVb crystal of compound (Bh) is subjected to DSC measurement, the enthalpy of the endothermic peak is about 124.3 J/g, and the extrapolated onset temperature is 62.3±5.0° C.

Here, the “extrapolated onset temperature” means, as defined in JIS K 7121 (plastic transition temperature measurement method), a temperature at the intersection of, in a DSC curve, an extrapolation baseline on the low temperature side heading toward a high temperature side, and a tangent line drawn at the point where a curve gradient on the low temperature side on the leading edge of the melting peak reaches maximum. When the enthalpy of the endothermic peak and an extrapolated onset temperature are within the above-mentioned range, the compound of the present invention has high stability.

The crystal of compound (Ah) of the present invention may be either Form I crystal or Form V crystal, or a crystal form mixture containing Form I crystal and/or Form V crystal. For the use of compound A as a pharmaceutical product and the like, Form I crystal or Form V crystal of compound (Ah), which is 0.5 hydrate of compound A, is preferable since it is a stable form crystal, and Form I crystal of compound (Ah) is more preferable since it is the most stable crystal form.

Moreover, the crystal of compound (Bh) of the present invention may be either Form IVb crystal or a crystal form mixture containing Form IVb crystal. For the use of compound B as a pharmaceutical product and the like, Form IVb crystal of compound (Bh), which is 2 hydrate of compound B, is preferable since it is a stable form crystal, and Form IVb crystal of compound (Bh) is more preferable since it is the most stable crystal form.

In the present invention, the “purity of crystal” means the ratio (purity) of compound (Ah) in a particular crystal form relative to the total amount of the crystal of compound (Ah).

In the present invention, the “purity of crystal” means the ratio (purity) of compound (Bh) in a particular crystal form relative to the total amount of the crystal of compound (Bh).

The purity of crystal of the present invention can be determined by a known method such as powder X-ray diffraction measurement method, thermoanalysis and the like. The purity of the crystal or crystal form mixture of the present invention is not necessarily be 100%, and is not less than 70%, preferably not less than 80%, more preferably not less than 90%, further preferably not less than 95%, most preferably not less than 98%. A purity within this range is preferable for guaranteeing the quality as a pharmaceutical product.

The compound of the present invention may be labeled with one or more isotopes (e.g., ³H, ²H, ¹⁴C, ³⁵S etc.).

For example, a deuterated form of compound (Ah) wherein any one or more ¹H are converted to ²H(D) is also encompassed in the compound of the present invention. Also, for example, a deuterated form of compound (Bh) wherein any one or more ¹H are converted to ²H(D) is also encompassed in the compound of the present invention.

As the “pharmaceutical composition”, a mixture of one or more pharmaceutically active ingredients and one or more kinds of pharmaceutically acceptable carriers, for example, oral preparations such as tablet, capsule, granule, fine granule, powder, troche, syrup, emulsion, suspension and the like, and parenteral agents such as external preparation, suppository, injection, eye drop, nasal preparation, pulmonary preparation and the like can be mentioned. Preferred is an oral preparation.

The pharmaceutical composition of the present invention is produced according to a method known per se in the art of pharmaceutical preparations, by mixing the compound of the present invention or a crystal thereof with a suitable amount of at least one kind of pharmaceutically acceptable carrier and the like as appropriate. While the content of the compound of the present invention or a crystal thereof in the pharmaceutical composition varies depending on the dosage form, dose and the like, it is, for example, 0.1 to 100 wt % of the whole composition. It is preferably 0.1 to 70 wt %.

Examples of the “pharmaceutically acceptable carrier” include various organic or inorganic carrier substances conventionally used as preparation materials, for example, excipient, disintegrant, binder, glidant, lubricant and the like for solid preparations, and solvent, solubilizing agent, suspending agent, isotonic agent, buffering agent, soothing agent and the like for liquid preparations. Where necessary, moreover, additives such as preservative, antioxidant, colorant, sweetening agent and the like are used.

Examples of the “excipient” include lactose, sucrose, D-mannitol, D-sorbitol, cornstarch, dextrin, crystalline cellulose, crystalline cellulose, carmellose, carmellose calcium, sodium carboxymethyl starch, low-substituted hydroxypropylcellulose, gum arabic and the like.

Examples of the “disintegrant” include carmellose, carmellose calcium, carmellose sodium, sodium carboxymethyl starch, croscarmellose sodium, crospovidone, low-substituted hydroxypropylcellulose, hydroxypropylmethylcellulose, crystalline cellulose and the like.

Examples of the “binder” include hydroxypropylcellulose, hydroxypropylmethylcellulose, povidone, crystalline cellulose, sucrose, dextrin, starch, gelatin, carmellose sodium, gum arabic and the like.

Examples of the “glidant” include light anhydrous silicic acid, magnesium stearate and the like.

Examples of the “lubricant” include magnesium stearate, calcium stearate, talc and the like.

Examples of the “solvent” include purified water, ethanol, propylene glycol, macrogol, sesame oil, corn oil, olive oil and the like.

Examples of the “solubilizing agents” include propylene glycol, D-mannitol, benzyl benzoate, ethanol, triethanolamine, sodium carbonate, sodium citrate and the like.

Examples of the “suspending agent” include benzalkonium chloride, carmellose, hydroxypropylcellulose, propylene glycol, povidone, methylcellulose, glycerol monostearate and the like.

Examples of the “isotonic agent” include glucose, D-sorbitol, sodium chloride, D-mannitol and the like.

Examples of the “buffering agent” include sodium hydrogenphosphate, sodium acetate, sodium carbonate, sodium citrate and the like.

Examples of the “soothing agent” include benzyl alcohol and the like.

Examples of the “preservative” include methyl parahydroxybenzoate, ethyl parahydroxybenzoate, propyl parahydroxybenzoate, chlorobutanol, benzyl alcohol, sodium dehydroacetate, sorbic acid and the like.

Examples of the “antioxidant” include sodium sulfite, ascorbic acid and the like.

Examples of the “colorant” include food colors (e.g., Food Color Red No. 2 or 3, Food Color yellow No. 4 or 5 etc.), β-carotene and the like.

Examples of the “sweetening agent” include saccharin sodium, dipotassium glycyrrhizinate, aspartame and the like.

The pharmaceutical composition of the present invention can be administered orally or parenterally (e.g., topical, intramuscular, subcutaneous, rectal, intravenous administration etc.) to human as well as mammals other than human (e.g., mouse, rat, hamster, guinea pig, rabbit, cat, dog, swine, bovine, horse, sheep, monkey etc.). The dose varies depending on the subject of administration, disease, symptom, dosage form, administration route and the like. For example, the daily dose for oral administration to an adult patient (body weight: about 60 kg) is generally within the range of about 1 mg to 1 g, based on the compound of the present invention as the active ingredient. This amount can be administered in one to several portions.

The compound of the present invention has a pyruvate dehydrogenase kinase (PDHK, i.e., PDHK1 and/or PDHK2) inhibitory activity and can effectively activate pyruvate dehydrogenase (PDH). Therefore, the compound of the present invention can be used as an active ingredient of a therapeutic agent or prophylactic agent for diabetes, insulin resistance syndrome, metabolic syndrome, hyperglycemia, hyperlacticacidemia, diabetic complications, cardiac failure, cardiomyopathy, myocardial ischemia, myocardial infarction, angina pectoris, dyslipidemia, atherosclerosis, peripheral artery disease, intermittent claudication, chronic obstructive pulmonary diseases, brain ischemia, cerebral apoplexy, mitochondrial disease, mitochondrial encephalomyopathy, cancer, pulmonary hypertension or Alzheimer disease.

Diabetes is, for example, type 1 diabetes or type 2 diabetes.

Examples of the diabetic complications include diabetic neuropathy, diabetic retinopathy, diabetic nephropathy and cataract.

Cardiac failure is, for example, acute cardiac failure or chronic cardiac failure.

To “inhibit PDHK” means to inhibit the function of PDHK and eliminate or attenuate the activity. To “inhibit PDHK”, human PDHK is preferably inhibited. As a “PDHK inhibitor”, preferred is a “human PDHK inhibitor”.

To “inhibit PDHK1” means to inhibit the function of PDHK1 and eliminate or attenuate the activity. For example, it means to inhibit the function as PDHK1 based on the conditions in the below-mentioned Experimental Example 1. To “inhibit PDHK1”, human PDHK1 is preferably inhibited. As a “PDHK1 inhibitor”, preferred is a “human PDHK1 inhibitor”. More preferred is a “PDHK1 inhibitor for human target organ”.

To “inhibit PDHK2” means to inhibit the function of PDHK2 and eliminate or attenuate the activity. For example, it means to inhibit the function as PDHK2 based on the conditions in the below-mentioned Experimental Example 1. To “inhibit PDHK2”, human PDHK2 is preferably inhibited. As a “PDHK2 inhibitor”, preferred is a “human PDHK2 inhibitor”. More preferred is a “PDHK2 inhibitor for human target organ”.

To “activate PDH” means to activate PDH in a target organ (e.g., liver, skeletal muscle, adipose tissue, heart, brain) and the like, cancer or the like.

To “decrease blood glucose level” means to decrease the glucose concentration in blood (including in serum and plasma), preferably to decrease high blood glucose level, more preferably, to decrease the blood glucose level to a therapeutically effective normal level for human.

To “decrease lactic acid level” means to decrease the lactic acid concentration in blood (including in serum and plasma), preferably to decrease high lactic acid level, more preferably, to decrease the lactic acid level to a therapeutically effective normal level for human.

The compound of the present invention can be used in combination with one or a plurality of other medicaments (hereinafter to be also referred to as a concomitant drug) according to a method generally employed in the medical field (hereinafter to be referred to as combined use).

The administration period of the compound of the present invention and a concomitant drug is not limited, and they may be administered to an administration subject as combination preparation, or the both preparations may be administered simultaneously or at given intervals. In addition, the pharmaceutical composition of the present invention and a concomitant drug may be used as a medicament in the form of a kit. The dose of the concomitant drug is similar to the clinically-employed dose and can be appropriately selected according to the subject of administration, disease, symptom, dosage form, administration route, administration time, combination and the like. The administration form of the concomitant drug is not particularly limited, and it only needs to be combined with the compound of the present invention.

Examples of the combination drug include therapeutic agents and/or prophylaxis agents for diabetes (type 1 diabetes, type 2 diabetes etc.), insulin resistance syndrome, metabolic syndrome, hyperglycemia, hyperlactacidemia, diabetic complications (diabetic neuropathy, diabetic retinopathy, diabetic nephropathy, cataract), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension or Alzheimer disease, and the like, and one or more agents therefrom and the compound of the present invention can be used in combination.

Examples of the “agent for the treatment and/or prophylaxis of diabetes” include insulin preparation, sulfonylurea hypoglycemic agent, metformin, DPP-4 inhibitor, thiazolidine derivative, GLP-1 receptor agonist and the like.

### EXAMPLES

While one embodiment of the production method of the compound of the present invention and crystal thereof is explained in the following, these Examples, Formulation Examples and Experimental Examples are mere exemplifications and do not limit the present invention.

Even if no description is found in the present production method, steps may be modified for efficient production, such as introduction of a protecting group into a functional group where necessary with deprotection in a subsequent step; using a functional group as a precursor in each step, followed by conversion to a desired functional group at a suitable stage; changing the order of production methods and steps, and the like.

The treatment after reaction in each step may be performed by a conventional method, where isolation and purification can be performed as necessary according to a method appropriately selected from conventional methods such as crystallization, recrystallization, distillation, partitioning, silica gel chromatography, preparative HPLC and the like, or a combination thereof.

All reagents and solvents have quality of commercially available products, and were used without further purification.

The powder X-ray diffraction analysis was performed using powder X-ray diffraction apparatus (X'Pert Pro, manufactured by Spectris Company).

Differential scanning calorimetry was performed using a differential scanning calorimetry (DSC) apparatus (DSC-60A, manufactured by SHIMADZU CORPORATION), or an apparatus for simultaneous measurements of powder X-ray diffraction and DSC thermogram (XRD-DSC) (XRD:RINT-2100; DSC:DSC8230, manufactured by Rigaku Corporation).

Water adsorption and desorption test was performed using a water equilibrium measuring apparatus (SGA-100, manufactured by VTI).

Differential heat/thermogravimetry simultaneous measurement (TG-DTA) was performed using a TG-DTA measuring apparatus (TGA/SDTA851^{e}/SF, manufactured by Mettler Toledo International Inc.).

Elemental analysis was performed using an elemental analysis apparatus (VarioELIII, manufactured by Elementar Analysensysteme GmbH).

Melting point measurement was performed using a melting point measurement device (Yanaco MP-500D, manufactured by Yanagimoto Mfg. Co., Ltd.).

Percentage % shows % by volume for the solvents used for chromatography, and wt % for others. Abbreviations used in other parts of the Examples mean the following.

s: singlet

d: doublet

t: triplet

q: quartet

m: multiplet

br: broad

J: coupling constant

CDCl₃: deuterated chloroform

DMSO-D₆: deuterated dimethyl sulfoxide

¹H NMR: proton nuclear magnetic resonance

HPLC: high performance liquid chromatography

¹H-NMR spectrum was measured in CDCl₃ or DMSO-D₆ using tetramethylsilane as an internal standard, and all δ values are shown in ppm.

### (Inducing Method A for Optical Purity Determination)

The solid (0.002 g-0.003 g) to be analyzed was shaken together with ethyl acetate (0.1 mL) and 1N hydrochloric acid (0.1 mL), after which the mixture was stood to allow for partitioning. The upper layer (0.010 mL) was added to the following preparation liquid (0.1 mL), and the mixture was shaken at 50° C. for 30 min. The obtained mixture was diluted with 50 v/v % acetonitrile water to 1 ml, and analyzed by HPLC.

### (Preparation Liquid)

Dimethylformamide was added to 1-(3-dimethylaminopropyl)-3-ethylcarbodiimide hydrochloride (0.191 g) and 1-hydroxybenzotriazole hydrate (0.153 g) to the total amount of 10 ml. To this mixture was added (1S)-1-phenylethylamine (0.258 ml) to give the title preparation liquid.

### (10 mM Phosphate Buffer (pH 2.0))

Potassium dihydrogen phosphate (4.08 g) was dissolved in water (3000 ml), and adjusted to pH 2.0 with phosphoric acid to give the title buffer.

### HPLC Analysis Conditions

Analysis condition 1
Measurement device: HPLC system SHIMADZU CORPORATION high-performance liquid chromatograph Prominence
Column: DAICEL CHIRALCEL OD-RH 4.6 mmΦ×150 mm
Column temperature: 40° C.
Mobile phase: (SOLUTION A) 10 mM phosphate buffer (pH 2.0), (SOLUTION B) acetonitrile
Fed while linearly changing the composition from SOLUTION A:SOLUTION B=50:50 to 20:80 over 20 min. Thereafter, fed for 5 min while maintaining at SOLUTION A:SOLUTION B=20:80.
Flow rate: 0.5 mL/min

### Detection: UV (220 nm)

### Example 1

### Synthesis of 2-{4-[(9R)-2-fluoro-9-hydroxy-9-(trifluoromethyl)-9H-fluoren-4-yl]-1H-pyrazol-1-yl}-2-methylpropane-1,3-diol (compound A)

### Step 1

### Ethyl 2′-chloro-4′-fluorobiphenyl-2-carboxylate

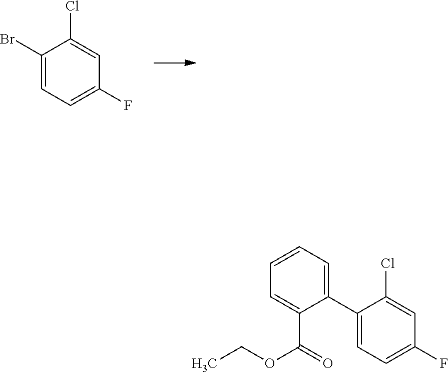 [SMILES: Fc1ccc(Br)c(Cl)c1 ; CCOC(=O)c1ccccc1-c1ccc(F)cc1Cl]

To a reaction vessel were added 1-bromo-2-chloro-4-fluorobenzene (25 g), ethyl 2-(4,4,5,5-tetramethyl[1,3,2]dioxaborolan-2-yl)benzoate (46 g), toluene (125 ml), water (125 ml) and tripotassium phosphate (50.5 g), and the reaction vessel was substituted with argon. To this mixture was added dichlorobis(triphenylphosphine)palladium(II) (1.67 g) and the mixture was stirred at an oil bath temperature 110° C. for 3 hr. The reaction vessel was removed from the oil bath, and water (125 ml) was added to the reaction mixture. The mixture was stirred at room temperature for 1 hr and filtered through celite. The filtrate was partitioned by pouring into a separating funnel. The aqueous layer was extracted with toluene, and combined with the organic layer. The organic layer was washed twice with water (125 ml), dried over anhydrous magnesium sulfate and filtered. The filtrate was concentrated under reduced pressure to give the title compound (41.8 g). The obtained solid was used for the next reaction without further purification.

¹H-NMR (400 MHz, CDCl₃) δ: 8.06-8.02 (1H, m), 7.60-7.54 (1H, m), 7.52-7.45 (1H, m), 7.27-7.16 (3H, m), 7.06-7.00 (1H, m), 4.18-4.09 (2H, m), 1.11-1.06 (3H, m).

### Step 2

### 2′-Chloro-4′-fluorobiphenyl-2-carboxylic acid

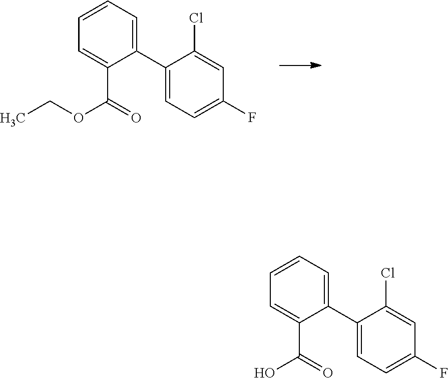 [SMILES: CCOC(=O)c1ccccc1-c1ccc(F)cc1Cl ; O=C(O)c1ccccc1-c1ccc(F)cc1Cl]

To a mixture of ethyl 2′-chloro-4′-fluorobiphenyl-2-carboxylate (41.8 g) and ethanol (179 ml) was added a 2N aqueous sodium hydroxide solution (179 ml), and the mixture was stirred for 2 hr at an oil bath temperature 80° C. The reaction mixture was cooled to room temperature, activated carbon (2.5 g) was added, and the mixture was stirred for 2.5 hr. The activated carbon was filtered off through celite and washed with 50 v/v % ethanol water (100 ml). The filtrate was acidified with 2N hydrochloric acid (196 ml). Water (33 ml) was added to the mixture, and the mixture was stirred at room temperature for 2 hr. The suspension was filtered, and the obtained solid was air-dried for 2 hr, and dried under reduced pressure at 60° C. to give the title compound (28.6 g).

¹H-NMR (400 MHz, CDCl₃) δ: 8.12-8.08 (1H, m), 7.64-7.59 (1H, m), 7.52-7.47 (1H, m), 7.27-7.24 (1H, m), 7.22-7.16 (2H, m), 7.05-7.00 (1H, m).

### Step 3

### 4-Chloro-2-fluoro-9H-fluoren-9-one

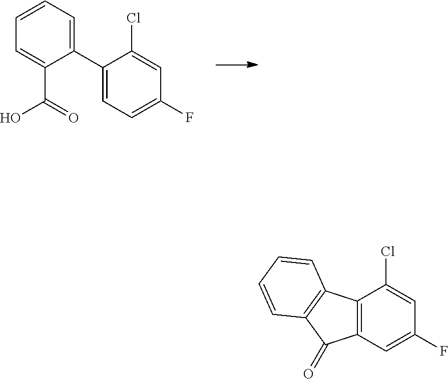 [SMILES: O=C(O)c1ccccc1-c1ccc(F)cc1Cl ; O=C1c2ccccc2-c2c(Cl)cc(F)cc21]

To a mixture of phosphorus pentoxide (V) (133 g) and methanesulfonic acid (1300 ml) was added 2′-chloro-4′-fluorobiphenyl-2-carboxylic acid (132.9 g), and the mixture was stirred at 80° C. for 2.5 hr. The reaction mixture was ice-cooled, water (1300 ml) was slowly added dropwise, and the mixture was further stirred at room temperature for 1 hr. The suspension was filtered, and the obtained solid was washed with water (300 ml). The solid was mixed with 50 v/v % ethanol water (1300 ml), and the mixture was slurry-washed (suspension stirred) at room temperature for 1.5 hr, and filtered. The obtained solid was washed with 50 v/v % ethanol water (200 ml), air-dried for 3 hr, and dried under reduced pressure at 60° C. to give the title compound (121.6 g).

¹H-NMR (400 MHz, CDCl₃) δ: 8.13-8.10 (1H, m), 7.72-7.69 (1H, m), 7.57-7.53 (1H, m), 7.36-7.30 (2H, m), 7.20-7.17 (1H, m).

### Step 4

### Ethyl[4-chloro-2-fluoro-9-(trifluoromethyl)-9H-fluoren-9-yloxy]acetate

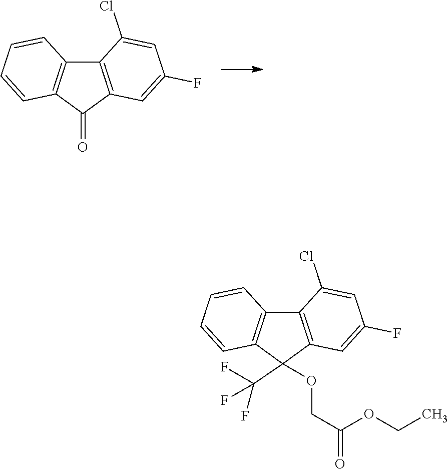 [SMILES: O=C1c2ccccc2-c2c(Cl)cc(F)cc21 ; CCOC(=O)COC1(C(F)(F)F)c2ccccc2-c2c(Cl)cc(F)cc21]

To a mixture of 4-chloro-2-fluoro-9H-fluoren-9-one (204 g) and dimethylformamide (1000 ml) was added potassium carbonate (36.4 g), and the mixture was stirred in a water bath. Trimethyl(trifluoromethyl)silane (156 ml) was added dropwise to the mixture over 30 min, and the mixture was further stirred at room temperature for 30 min. Cesium fluoride (173 g) was added to the reaction mixture, then ethyl bromoacetate (75 ml) was added dropwise over 20 min, and the mixture was further stirred at room temperature. Water (1000 ml) was added to the reaction mixture, and the mixture was poured into a separating funnel m and extracted with ethyl acetate (1000 ml). The organic layer was washed twice with brine (water:saturated brine=4:1, 1000 ml) and once with saturated brine (500 ml). The obtained organic layer was dried over anhydrous sodium sulfate, and filtered. The filtrate was concentrated under reduced pressure to give the title compound (360 g). The obtained residue was directly used for the next reaction without further purification.

¹H-NMR (400 MHz, CDCl₃) δ: 8.30-8.27 (1H, m), 7.73-7.69 (1H, m), 7.57-7.52 (1H, m), 7.43-7.37 (2H, m), 7.25-7.21 (1H, m), 4.11 (2H, q, J=7.1 Hz), 3.60 (1H, d, J=15.5 Hz), 3.53 (1H, d, J=15.3 Hz), 1.19 (3H, t, J=7.2 Hz).

### Step 5

### [4-Chloro-2-fluoro-9-(trifluoromethyl)-9H-fluoren-9-yloxy]acetic acid

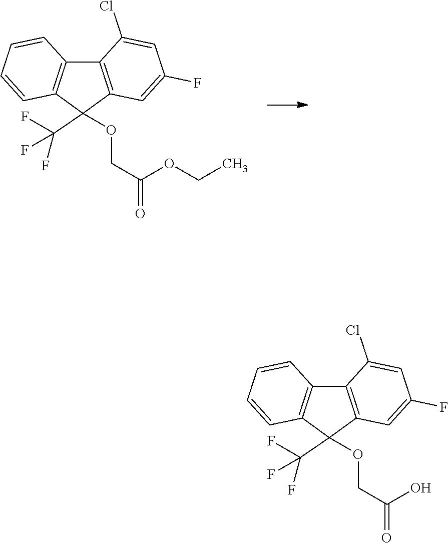 [SMILES: CCOC(=O)COC1(C(F)(F)F)c2ccccc2-c2c(Cl)cc(F)cc21 ; O=C(O)COC1(C(F)(F)F)c2ccccc2-c2c(Cl)cc(F)cc21]

To a mixture of ethyl[4-chloro-2-fluoro-9-(trifluoromethyl)-9H-fluoren-9-yloxy]acetate (360 g) and ethanol (440 ml) was added 2N aqueous sodium hydroxide solution (877 ml), and the mixture was stirred at 80° C. for 3.5 hr. The reaction mixture was cooled to room temperature, and the insoluble material was filtered off through celite and washed with water (500 ml) and ethanol (60 ml). Water (120 ml) was added to the filtrate, the mixture was ice-cooled, and formic acid (199 ml) was added dropwise. The suspension was stirred at room temperature overnight and filtered. The obtained solid was washed with 25 v/v % ethanol water (400 ml), air-dried overnight, and dried under reduced pressure at 60° C. to give the title compound (285 g).

¹H-NMR (400 MHz, CDCl₃) δ: 8.32-8.29 (1H, m), 7.71-7.67 (1H, m), 7.59-7.54 (1H, m), 7.45-7.40 (1H, m), 7.38-7.34 (1H, m), 7.27-7.23 (1H, m), 3.65 (1H, d, J=16.0 Hz), 3.60 (1H, d, J=16.0 Hz).

### Step 6

### (1R)-1-(1-Naphthyl)ethylamine salt of [(9R)-4-chloro-2-fluoro-9-(trifluoromethyl)-9H-fluoren-9-yloxy]acetic acid

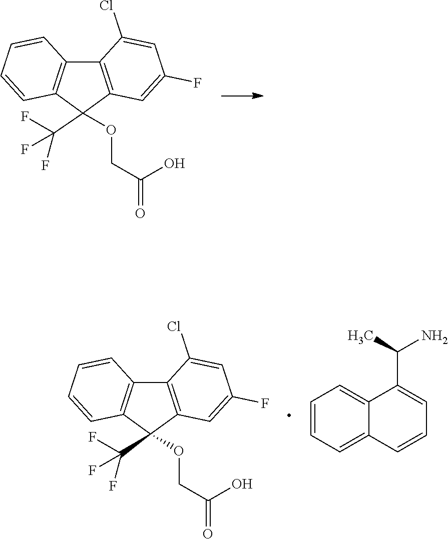

To a mixture of [4-chloro-2-fluoro-9-(trifluoromethyl)-9H-fluoren-9-yloxy]acetic acid (50 g) and methyl ethyl ketone (250 ml) was added (1R)-1-(1-naphthyl)ethylamine (11.1 mL), and the mixture was stirred at 50° C. for 3 days. The suspension was cooled to room temperature, further stirred for 4 days, and filtered. The obtained solid was dried under reduced pressure to give the title compound (25.5 g). The solid was subjected to inducing method A for optical purity determination and analyzed under HPLC analysis condition 1. It contained a large amount of (R)-form carboxylic acid derivatives, and the optical purity was 92.5% e.e.

(S)-form carboxylic acid derivative (retention time 22.58 min)
(R)-form carboxylic acid derivative (retention time 22.73 min)

¹H-NMR (400 MHz, DMSO-D₆) δ: 8.27 (1H, d, J=7.7 Hz), 8.26 (3H, br s), 8.14 (1H, d, J=8.4 Hz), 8.00-7.94 (1H, m), 7.89 (1H, d, J=8.4 Hz), 7.73 (1H, d, J=7.7 Hz), 7.70-7.47 (8H, m), 5.12 (1H, q, J=6.6 Hz), 3.19 (1H, d, J=14.3 Hz), 3.14 (1H, d, J=14.3 Hz), 1.52 (3H, d, J=6.6 Hz).

### Step 7

### [(9R)-4-Chloro-2-fluoro-9-(trifluoromethyl)-9H-fluoren-9-yloxy]acetic acid

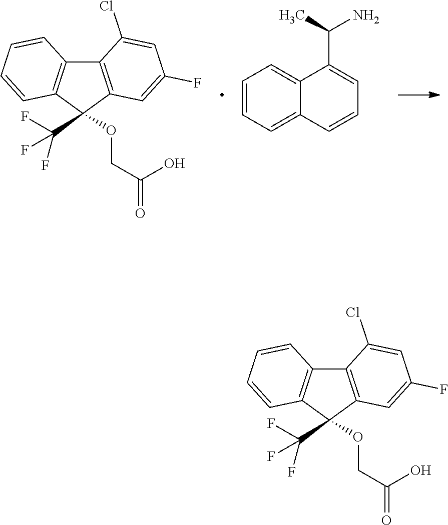

To a mixture of (1R)-1-(1-naphthyl)ethylamine salt of [(9R)-4-chloro-2-fluoro-9-(trifluoromethyl)-9H-fluoren-9-yloxy]acetic acid (25.45 g) and ethyl acetate (178 ml) were added 2N hydrochloric acid (51 ml) and water (127 ml), and the mixture was stirred at room temperature for 1 hr. The mixture was poured into a separating funnel and partitioned. The organic layer was washed twice with water (100 ml) and with saturated brine (100 ml). The obtained organic layer was dried over anhydrous sodium sulfate, and filtered. The filtrate was concentrated under reduced pressure, hexane (127 ml) was added to the obtained residue, and the mixture was slurry-washed (suspension stirred) at room temperature for 1 hr. The suspension was filtered, and the obtained solid was washed with hexane and dried under reduced pressure to give the title compound (16.33 g).

¹H-NMR (400 MHz, DMSO-D₆) δ: 12.78 (1H, br s), 8.31-8.28 (1H, m), 7.73-7.65 (3H, m), 7.56-7.49 (2H, m), 3.57 (1H, d, J=15.8 Hz), 3.51 (1H, d, J=15.5 Hz).

### Step 8

### (9R)-4-Chloro-2-fluoro-9-(trifluoromethyl)-9H-fluoren-9-ol

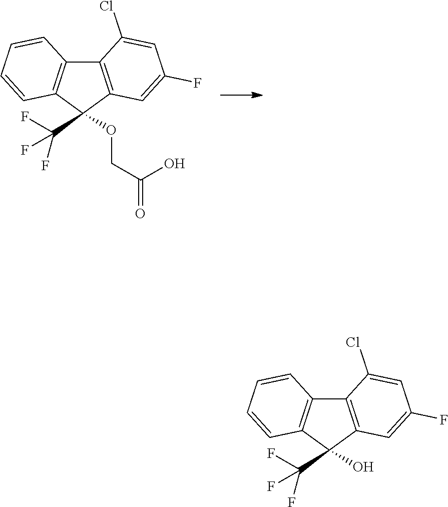 [SMILES: O=C(O)CO[C@]1(C(F)(F)F)c2ccccc2-c2c(Cl)cc(F)cc21 ; O[C@]1(C(F)(F)F)c2ccccc2-c2c(Cl)cc(F)cc21]

To a mixture of [(9R)-4-chloro-2-fluoro-9-(trifluoromethyl)-9H-fluoren-9-yloxy]acetic acid (36.74 g) and dimethylformamide (184 ml) was added N-ethyldiisopropylamine (20.9 mL), and the mixture was stirred at 0° C. Diphenylphosphoryl azide (23.7 mL) was added dropwise thereto over 30 min, and the mixture was further stirred at 0° C. for 2 hr. Acetic acid (2.86 ml) was added to the reaction mixture, and the mixture was heated to room temperature. t-Butyl alcohol (96 ml) was added, and the mixture was stirred at 100° C. for 1 hr. The reaction mixture was ice-cooled, 2N hydrochloric acid (367 ml) was added, and the mixture was allowed to cool to room temperature and stirred overnight. The reaction solution was poured into a separating funnel and extracted three times with toluene (180 ml). The combined organic layer was successively washed with water (180 ml), 1N aqueous sodium hydroxide solution (180 ml), water (180 ml) and saturated brine (180 ml). The obtained organic layer was dried over anhydrous sodium sulfate, and filtered. The filtrate was concentrated under reduced pressure, the obtained residue, ethanol (37 ml), tetrahydrofuran (37 ml) and 2N aqueous sodium hydroxide solution (37 ml) were mixed, and the mixture was stirred at 60° C. for 3 hr. The reaction mixture was cooled to room temperature, and water (180 ml) was added. The mixture was poured into a separating funnel and extracted twice with toluene (180 ml). The organic layer was washed twice with water (180 ml) and once with saturated brine (180 ml). The obtained organic layer was dried over anhydrous sodium sulfate, and filtered. The filtrate was concentrated under reduced pressure and the obtained residue was purified by silica gel column chromatography (a mixture of hexane and ethyl acetate was used as an elution solvent; the mixture was first eluted with hexane:ethyl acetate at a mixing ratio 9:1, and further at mixing ratio 8:2) to give the title compound (21.69 g). specific optical rotation [α]_{D}=+30.60° (20° C., c=1.00, methanol).

¹H-NMR (400 MHz, CDCl₃) δ: 8.29-8.26 (1H, m), 7.73-7.69 (1H, m), 7.55-7.50 (1H, m), 7.43-7.35 (2H, m), 7.21-7.18 (1H, m), 2.82 (1H, s).

### (Absolute Configuration)

4-Chloro-2-methyl-9H-fluoren-9-one obtained in Example 2, step 3, was subjected to trifluoromethylation to give [4-chloro-2-methyl-9-(trifluoromethyl)-9H-fluoren-9-yloxy]acetic acid. This compound was optically resolved using (1R)-1-phenylethylamine, and the absolute configuration of the obtained (1R)-1-phenylethylamine salt (compound 100AA) was determined to be (R) by single crystal X-ray structural analysis.

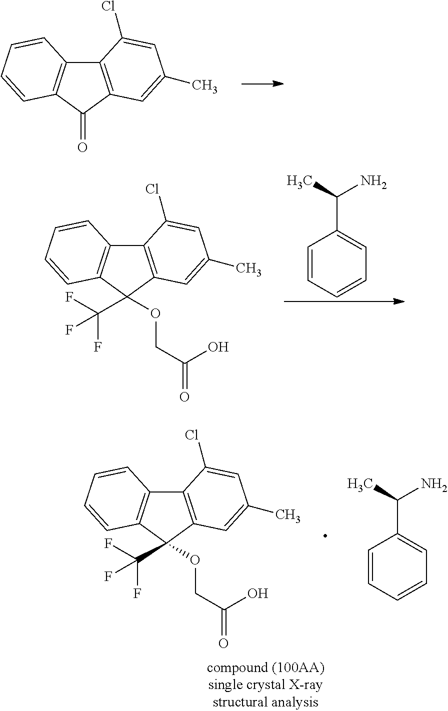

The absolute configuration of the fluorine compound (4-chloro-2-fluoro-9-(trifluoromethyl)-9H-fluoren-9-ol) derived from compound 100AA and the compound (4-chloro-2-fluoro-9-(trifluoromethyl)-9H-fluoren-9-ol) obtained in the aforementioned step 8 was determined by HPLC analysis using an optically active column.

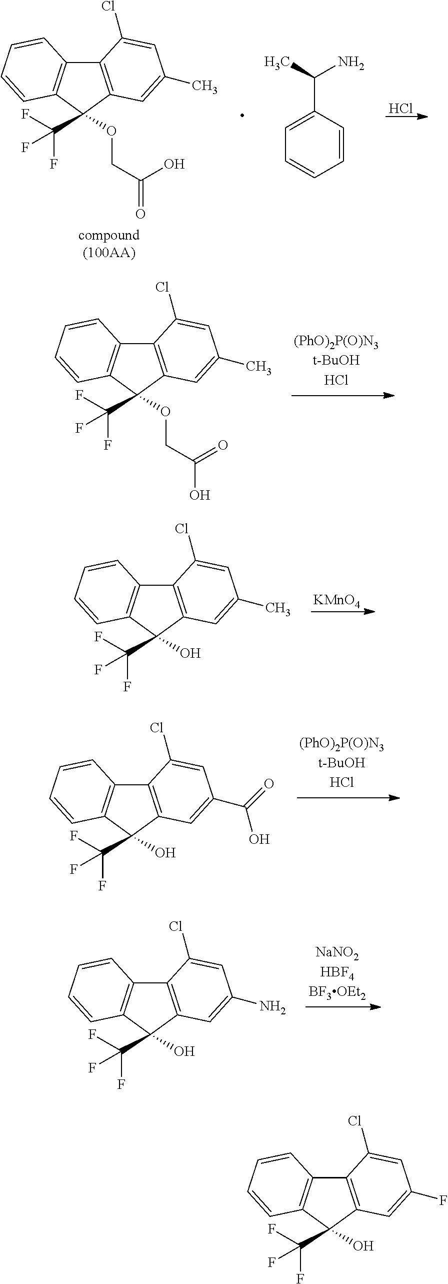

### Step 9

### Ethyl 2-[4-(4,4,5,5-tetramethyl[1,3,2]dioxaborolan-2-yl)-1H-pyrazol-1-yl]propionate

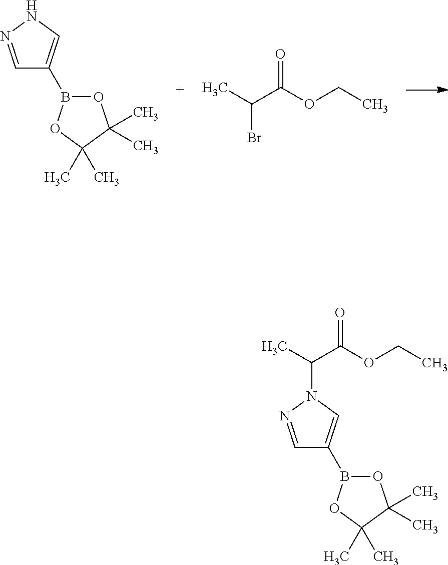

To a suspension of 4-(4,4,5,5-tetramethyl[1,3,2]dioxaborolan-2-yl)-1H-pyrazole (21.3 g) and potassium carbonate (20.7 g) in dimethylformamide (100 ml) was added ethyl 2-bromopropionate (13 ml), and the mixture was stirred at 80° C. for 14 hr. The reaction mixture was cooled to 0° C., and toluene (100 ml) and water (150 ml) were successively added dropwise. The mixture was partitioned, and the aqueous layer was extracted with toluene (50 ml). The combined organic layer was successively washed once with 10% aqueous potassium carbonate solution (50 ml), twice with water (50 ml) and once with saturated brine (50 ml). The obtained organic layer was dried over anhydrous sodium sulfate, and filtered. The filtrate was concentrated under reduced pressure to give the title compound (21.6 g).

¹H-NMR (400 MHz, CDCl₃) δ: 7.85 (1H, s), 7.81 (1H, s), 5.10 (1H, q, J=7.3 Hz), 4.19 (2H, q, J=7.1 Hz), 1.78 (3H, d, J=7.4 Hz), 1.32 (12H, s), 1.25 (3H, t, J=7.2 Hz).

### Step 10

### Ethyl 2-{4-[(9R)-2-fluoro-9-hydroxy-9-(trifluoromethyl)-9H-fluoren-4-yl]-1H-pyrazol-1-yl}propionate

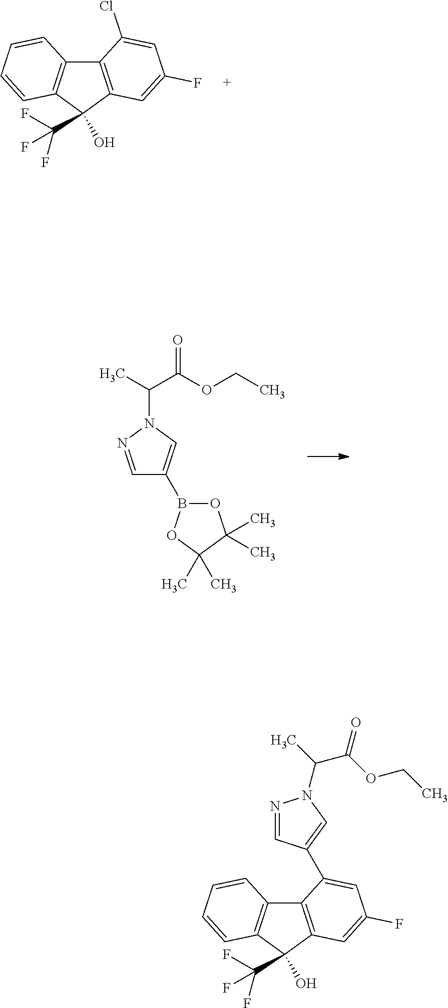 [SMILES: O[C@]1(C(F)(F)F)c2ccccc2-c2c(Cl)cc(F)cc21 ; CCOC(=O)C(C)n1cc(B2OC(C)(C)C(C)(C)O2)cn1 ; CCOC(=O)C(C)n1cc(-c2cc(F)cc3c2-c2ccccc2[C@]3(O)C(F)(F)F)cn1]

To a suspension of ethyl 2-[4-(4,4,5,5-tetramethyl[1,3,2]dioxaborolan-2-yl)-1H-pyrazol-1-yl]propionate (29.2 g), (9R)-4-chloro-2-fluoro-9-(trifluoromethyl)-9H-fluoren-9-ol (20.4 g) and sodium hydrogen carbonate (11.1 g) in toluene/water (200 ml/66 ml) were added palladium acetate (743 mg) and 2-dicyclohexylphosphino-2′,6′-dimethoxybiphenyl (2.72 g) at room temperature, and the mixture was stirred at 115° C. for 8 hr. The reaction mixture was allowed to cool to room temperature, activated carbon (10 g) and celite (10 g) were added, and the mixture was stirred for 1 hr. The mixture was filtered through celite, and the solid was washed with toluene (100 ml). The filtrate was partitioned, and the aqueous layer was extracted with toluene (60 ml). The combined organic layer was successively washed three times with water (100 ml) and once with saturated brine (100 ml). The organic layer was dried over anhydrous sodium sulfate, and filtered. The filtrate was concentrated under reduced pressure. To a solution of the obtained residue in toluene/ethyl acetate (3/1, 130 ml) was added silica gel (40 g), and the mixture was stirred at room temperature for 1 hr. The mixture was filtered, and the obtained filtrate was concentrated under reduced pressure. The obtained residue was purified by silica gel column chromatography (a mixture of hexane and ethyl acetate was used as an elution solvent; the mixture was first eluted with hexane:ethyl acetate at a mixing ratio 5:1, and further at mixing ratio 2:1) to give the title compound (27.9 g).

¹H-NMR (400 MHz, DMSO-D₆) δ: 8.20-8.18 (1H, m), 7.72-7.71 (1H, m), 7.67-7.63 (1H, m), 7.44-7.40 (2H, m), 7.37-7.23 (4H, m), 5.40-5.34 (1H, m), 4.22-4.15 (2H, m), 1.78-1.75 (3H, m), 1.23-1.18 (3H, m).

### Step 11

### 2-{4-[(9R)-2-Fluoro-9-hydroxy-9-(trifluoromethyl)-9H-fluoren-4-yl]-1H-pyrazol-1-yl}-3-hydroxy-2-methylpropionic acid

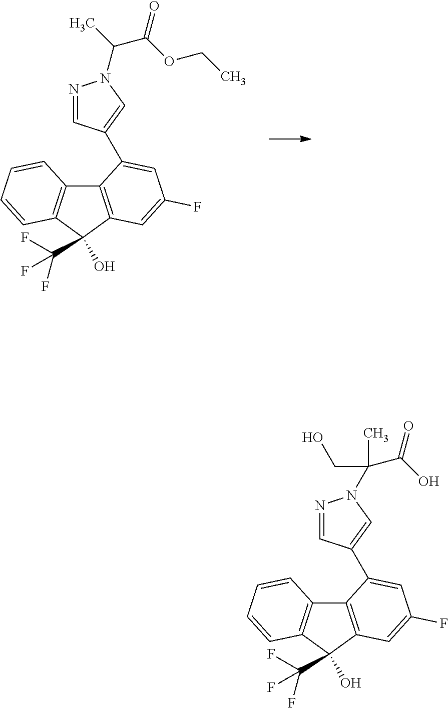 [SMILES: CCOC(=O)C(C)n1cc(-c2cc(F)cc3c2-c2ccccc2[C@]3(O)C(F)(F)F)cn1 ; CC(CO)(C(=O)O)n1cc(-c2cc(F)cc3c2-c2ccccc2[C@]3(O)C(F)(F)F)cn1]

To a solution of ethyl 2-{4-[(9R)-2-fluoro-9-hydroxy-9-(trifluoromethyl)-9H-fluoren-4-yl]-1H-pyrazol-1-yl}propionate (27.9 g) and paraformaldehyde (17.0 g) in dimethylformamide (100 ml) was added a 1M tetrahydrofuran solution (170 ml) of tetrabutylammonium fluoride at room temperature, and the mixture was stirred at 100° C. for 6 hr. The reaction mixture was filtered through celite, and the obtained solid was washed with ethyl acetate (100 ml). 1N Hydrochloric acid (400 ml) was added to the filtrate, and the mixture was extracted with ethyl acetate (100 ml). The separated aqueous layer was extracted twice with ethyl acetate (100 ml). The combined organic layer was successively washed once with 1N hydrochloric acid (100 ml), twice with brine (water/saturated brine=100 ml/10 ml), and once with saturated brine. The organic layer was dried over anhydrous sodium sulfate, and filtered. The filtrate was concentrated under reduced pressure. The obtained residue was azeotropically distilled twice with toluene to give the title compound (26.6 g).

¹H-NMR (400 MHZ, DMSO-D₆) δ: 8.19-8.17 (1H, m), 7.71-7.70 (1H, m), 7.66-7.61 (1H, m), 7.46-7.19 (6H, m), 5.31 (1H, brs), 4.21-4.12 (1H, m), 3.96-3.88 (1H, m), 1.80 (3H, s).

### Step 12

### 2-{4-[(9R)-2-Fluoro-9-hydroxy-9-(trifluoromethyl)-9H-fluoren-4-yl]-1H-pyrazol-1-yl}-2-methyl-propane-1,3-diol (compound A)

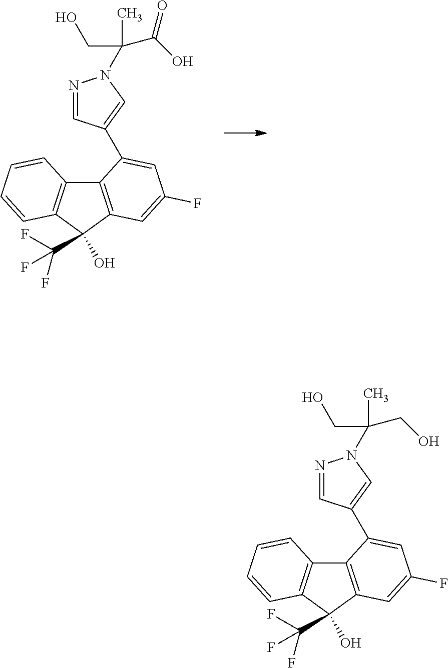 [SMILES: CC(CO)(C(=O)O)n1cc(-c2cc(F)cc3c2-c2ccccc2[C@]3(O)C(F)(F)F)cn1 ; CC(CO)(CO)n1cc(-c2cc(F)cc3c2-c2ccccc2[C@]3(O)C(F)(F)F)cn1]

To a solution of 2-{4-[(9R)-2-fluoro-9-hydroxy-9-(trifluoromethyl)-9H-fluoren-4-yl]-1H-pyrazol-1-yl}-3-hydroxy-2-methylpropionic acid (26.6 g) in tetrahydrofuran (40 ml) was added dropwise a 1.09M tetrahydrofuran solution (200 ml) of borane-tetrahydrofuran complex at room temperature, and the mixture was stirred for 3 hr. Ethanol (25 ml) was added dropwise to the reaction mixture at room temperature, and the mixture was stirred at 80° C. for 1 hr. Water (200 ml) and saturated aqueous sodium hydrogen carbonate solution (100 ml) were added to the mixture, and the mixture was extracted twice with ethyl acetate (100 ml). The combined organic layer was successively washed twice with water (100 ml) and once with saturated brine (100 ml). The obtained organic layer was dried over anhydrous sodium sulfate, and filtered. The filtrate was concentrated under reduced pressure. The obtained residue was purified by silica gel column chromatography (a mixture of chloroform and methanol was used as an elution solvent; the mixture was first eluted with chloroform:methanol at a mixing ratio 20:1, and further at mixing ratio 10:1) to give the title compound (17.4 g).

Specific optical rotation [α]_{D}=+72.4° (25° C., c=1.004, methanol).

¹H-NMR (400 MHZ, DMSO-D₆) δ: 8.05 (1H, d, J=0.7 Hz), 7.68 (1H, d, J=0.7 Hz), 7.65-7.62 (1H, m), 7.44-7.42 (1H, m), 7.40-7.36 (2H, m), 7.35-7.26 (2H, m), 7.21-7.17 (1H, m), 4.98-4.93 (2H, m), 3.84-3.79 (2H, m), 3.76-3.70 (2H, m), 1.52 (3H, s).

### Example 1-1

Compound A (50 mg) obtained in Example 1 was suspended in water (0.20 ml), and the suspension was stirred at room temperature overnight. The precipitated solid was collected by filtration, and dried under reduced pressure at room temperature to give 0.5 hydrate of compound A (compound (Ah)) as Form I crystal (34 mg).

Melting point 91-99° C.

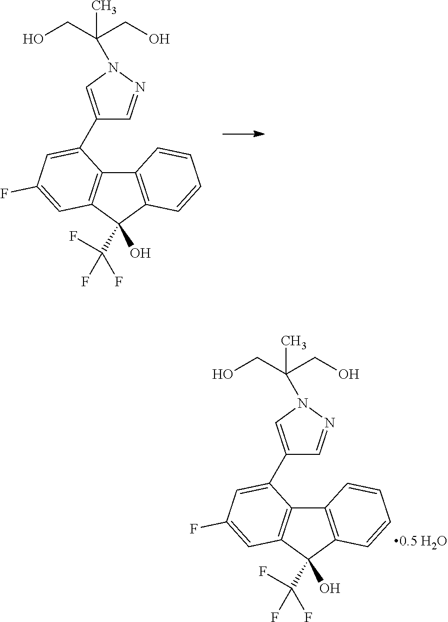 [SMILES: CC(CO)(CO)n1cc(-c2cc(F)cc3c2-c2ccccc2[C@]3(O)C(F)(F)F)cn1 ; CC(CO)(CO)n1cc(-c2cc(F)cc3c2-c2ccccc2[C@]3(O)C(F)(F)F)cn1]

### (Powder X-Ray Diffraction Measurement)

The Form I crystal of compound (Ah) was subjected to powder X-ray diffraction analysis under the following conditions.

### Conditions:

X-ray source: Cu—Kα line
Tube voltage: 45 kV
Tube electric current: 40 mA
Range of angle measurement: 2θ=3° to 25°

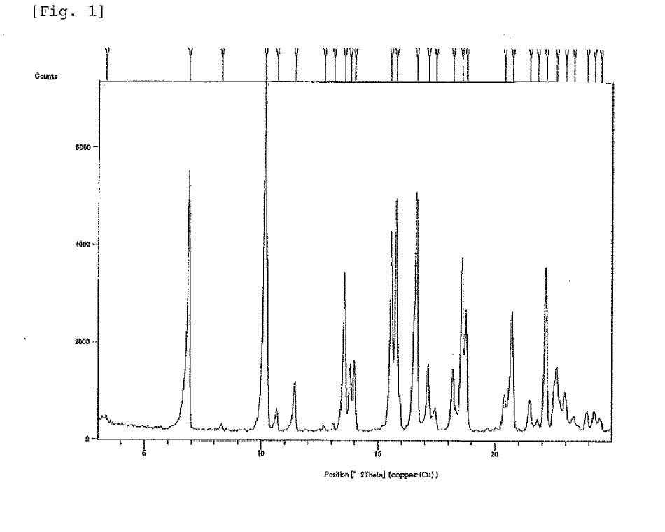

The powder X-ray diffraction pattern is shown in FIG. 1, and the relative intensity when the peak intensity of diffraction angle (2θ) of 10.2° is 100 is shown in Table 3.

TABLE 3

| | relative intensity | NET intensity |
|---|---|---|
| Pos. [°2Th.] | [%] | [cts] |
| 3.3317 | 2.77 | 200.57 |
| 6.8793 | 74.26 | 6386.14 |
| 8.2956 | 1.98 | 143.93 |
| 10.1647 | 100.00 | 7252.64 |
| 10.6533 | 6.35 | 460.64 |
| 11.4229 | 14.04 | 1018.53 |
| 12.6712 | 1.36 | 98.28 |
| 13.0816 | 1.96 | 141.80 |
| 13.5502 | 44.85 | 3252.99 |
| 13.7982 | 18.62 | 1350.56 |
| 13.9984 | 19.91 | 1444.07 |
| 15.5328 | 56.99 | 4133.20 |
| 15.7650 | 65.82 | 4773.94 |
| 16.6441 | 68.86 | 4994.32 |
| 17.1335 | 19.33 | 1401.80 |
| 17.4440 | 6.44 | 466.71 |
| 18.1837 | 17.95 | 1301.61 |
| 18.5774 | 47.73 | 3461.98 |
| 18.7787 | 32.67 | 2369.39 |
| 20.3793 | 10.30 | 746.72 |
| 20.7151 | 33.59 | 2436.49 |
| 21.4580 | 9.06 | 657.01 |
| 21.7939 | 3.43 | 248.99 |
| 22.1436 | 46.75 | 3390.28 |
| 22.6122 | 18.48 | 1339.95 |
| 22.9773 | 11.30 | 819.87 |
| 23.3168 | 4.15 | 301.26 |
| 23.8856 | 5.45 | 395.23 |
| 24.1980 | 5.71 | 414.43 |
| 24.4588 | 3.52 | 255.36 |

### (Differential Scanning Calorimetry/DSC)

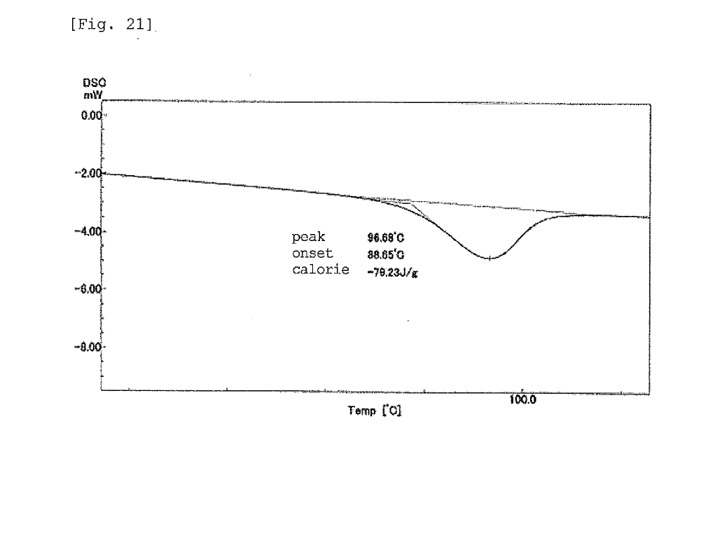

Form I crystal (2-3 mg) was measured using differential scanning calorimetry (DSC) apparatus DSC-60A (manufactured by SHIMADZU CORPORATION) at a temperature rise rate of 5° C./min (sealed aluminum pan). The DSC curve obtained by the measurement is shown in FIG. 21. The enthalpy of the endothermic peak on the DSC curve was about 79.2 J/g, and the extrapolated onset temperature was 88.7±5° C.

### (Differential Heat/Thermogravimetry Simultaneous Measurement)

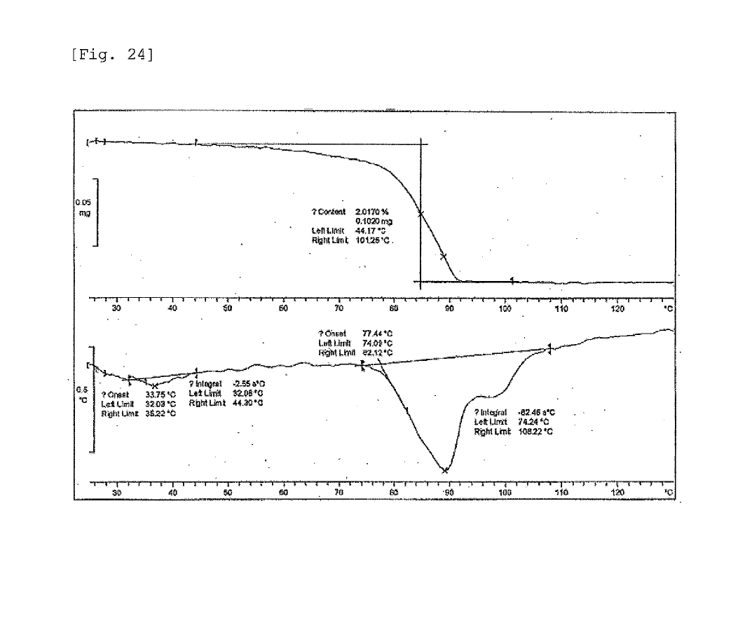

Form I crystal (about 5 mg) was placed in an open aluminum pan, and measured using TG-DTA measuring apparatus (TGA/SDTA851^{e}/SF, manufactured by Mettler Toledo International Inc.) under a dried nitrogen atmosphere at a temperature rise rate of 5° C./min. The measurement results are shown in FIG. 24.

According to FIG. 24, a weight loss of 2.02% was found by heating, and the decrease ratio thereof was well consistent with the theoretical water content of 2.09% of compound (Ah).

### (Elemental Analysis Measurement)

Furthermore, Form I crystal (about 2 mg) was wrapped with a tin foil, and measured using an elemental analysis apparatus (VarioELIII, manufactured by Elementar Analysensysteme GmbH). The results of the elemental analysis of Form I crystal were well consistent with the calculated values of compound (Ah), as shown below.

Calculated: C, 58.47; H, 4.44; N, 6.49 (calculated as 0.5 hydrate);

Found: C, 58.41; H, 4.50; N, 6.54.

### (Water Adsorption and Desorption Measurement)

A water adsorption and desorption test was performed by the following method.

Method 1 (without Pre-Drying):

Form I crystal (about 10 mg) was weighed in a quartz cell, and a weight change ratio was measured when it reached equilibrium at 25° C. and each relative humidity, by using a water equilibrium measuring apparatus (SGA-100, manufactured by VTI).

### Test Conditions:

Test temperature: 25° C.
Equilibrating conditions: weight change ratio was not more than 0.03% in 5 min
Maximum equilibrating time: 180 min
Change of relative humidity: increased from 5% to 95% at 5% intervals, and decreased from 95% to 5% at 5% intervals.
Method 2 (with Pre-Drying):

Form I crystal (about 10 mg) was weighed in a quartz cell, dried under a dried nitrogen stream at 60° C., and a weight change ratio was measured when it reached equilibrium at 25° C. and each relative humidity, by using a water equilibrium measuring apparatus (SGA-100, manufactured by VTI).

### Test Conditions:

Test temperature: 25° C.
Equilibrating conditions: weight change ratio was not more than 0.03% in 5 min
Maximum equilibrating time: 180 min
Change of relative humidity: increased from 5% to 95% at 5% intervals, and decreased from 95% to 5% at 5% intervals.

In the water adsorption and desorption test, the measurement was performed under two conditions of “with pre-drying (drying before test)” and “without pre-drying (drying before test)”, wherein humidification and drying were repeated once. As a result, when Form I crystal was pre-dried (under a dried nitrogen stream, 60° C.), the weight decreased by 2.15%, and the decrease ratio thereof was well consistent with the theoretical water content of 2.09% of compound (Ah). Therefore, it was assumed that pre-drying completely evaporated crystal water.

On the other hand, it is considered that 0.5 equivalent of water was reabsorbed by humidification, since humidification after drying up to relative humidity 10% resulted in a weight increase of 2.44%.

When pre-drying was not performed, remarkable moisture absorption was not found even under relative humidity 95%, and the rate of moisture absorption under relative humidity 75% was 0.275 to 0.282% and less than 3%. The results confirm that Form I crystal of compound (Ah) does not have hygroscopicity.

### Example 1-2

Compound A (50 mg) obtained in Example 1 was suspended in 30 v/v % methanol water (0.10 ml), and the mixture was stirred at room temperature for 1 hr. The precipitated solid was collected by filtration, and dried under reduced pressure at room temperature to give 0.5 hydrate of compound A (compound (Ah)) as Form V crystal (40 mg). Melting point 90-100° C.

### (Powder X-Ray Diffraction Analysis)

Form V crystal of compound (Ah) was subjected to powder X-ray diffraction analysis under conditions similar to those in Example 1-1.

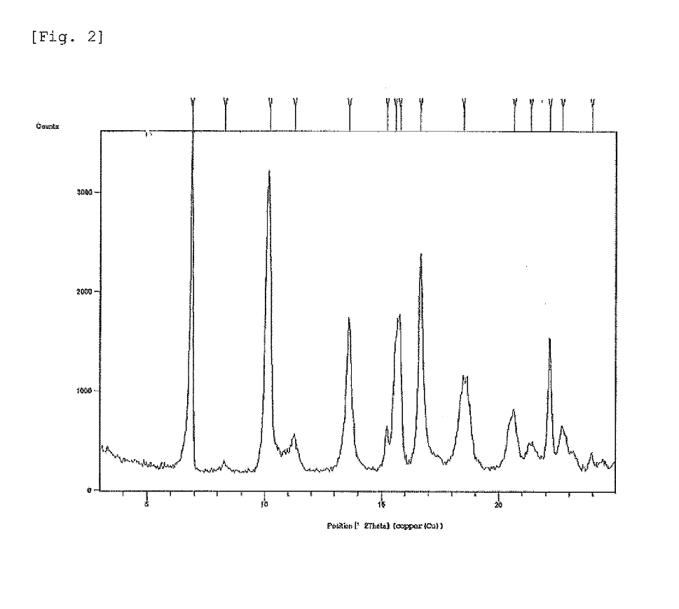

The powder X-ray diffraction pattern is shown in FIG. 2, and the relative intensity, wherein the peak intensity at diffraction angle 2θ of 6.9° is 100, is shown in Table 4.

TABLE 4

| | relative | NET |
|---|---|---|
| | intensity | intensity |
| Pos. [°2Th.] | [%] | [cts] |
| 6.8890 | 100.00 | 3402.32 |
| 8.2774 | 2.04 | 69.26 |
| 10.1874 | 86.09 | 2929.11 |
| 11.2672 | 11.07 | 376.55 |
| 13.5951 | 45.52 | 1548.60 |
| 15.1953 | 13.31 | 452.87 |
| 15.5414 | 36.23 | 1232.72 |
| 15.7693 | 45.84 | 1559.57 |
| 16.6431 | 64.73 | 2202.29 |
| 18.4655 | 28.41 | 966.54 |
| 20.6442 | 18.36 | 624.57 |
| 21.3386 | 8.46 | 287.96 |
| 22.1489 | 40.41 | 1374.94 |
| 22.6848 | 13.70 | 466.27 |
| 23.9240 | 5.57 | 189.57 |

### (Differential Heat/Thermogravimetry Simultaneous Measurement)

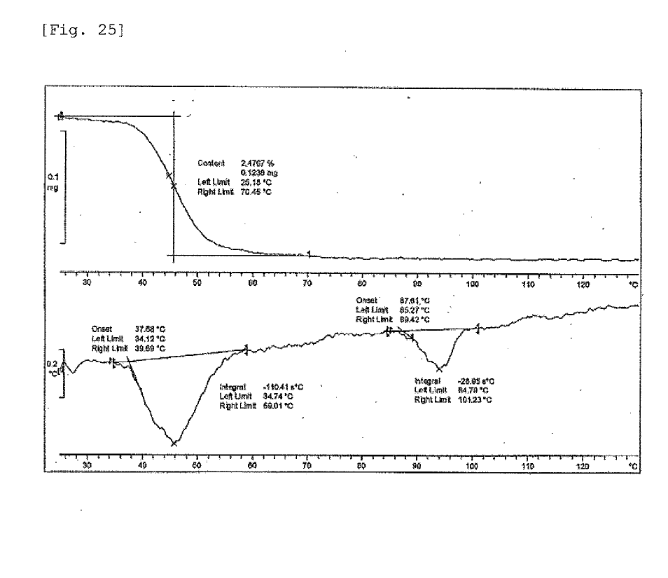

Form V crystal (about 5 mg) was placed in an open aluminum pan, and measured using TG-DTA measuring apparatus (TGA/SDTA851^{e}/SF, manufactured by Mettler Toledo International Inc.) under a dried nitrogen atmosphere at a temperature rise rate of 2° C./min. The measurement results are shown in FIG. 25.

According to FIG. 25, a weight loss of 2.48% was found by heating, and the decrease ratio thereof was well consistent with the theoretical water content of 2.09% of compound (Ah).

### (Elemental Analysis Measurement)

Furthermore, Form V crystal was subjected to elemental analysis under conditions similar to those in Example 1-1. The results of the elemental analysis of Form V crystal were well consistent with the calculated values of compound (Ah), as shown below.

Calculated: C, 58.47; H, 4.44; N, 6.49 (calculated as 0.5 hydrate);

Found: C, 58.30; H, 4.49; N, 6.54.

### (Water Adsorption and Desorption Measurement)

Form V crystal was subjected to water adsorption and desorption test under conditions similar to those in Example 1-1.

As a result, when pre-drying was performed (under dried nitrogen stream, 60° C.), the weight decreased by 2.36%, and the decrease ratio thereof was well consistent with the theoretical water content of 2.09% of compound (Ah). Therefore, it was assumed that pre-drying completely evaporated crystal water.

On the other hand, it is considered that 0.5 equivalent of water was reabsorbed by humidification, since humidification after drying up to relative humidity 10% resulted in a weight increase of 2.46%.

When pre-drying was not performed, remarkable moisture absorption was not found even under relative humidity 95%, and the rate of moisture absorption under relative humidity 75% was 0.210 to 0.237% and less than 3%. The results confirm that Form V crystal of compound (Ah) does not have hygroscopicity.

### Example 1-3

Compound A (957 mg) obtained in Example 1 was suspended in 30 v/v % ethanol water (2.0 ml), and the suspension was stirred at room temperature overnight. The precipitated solid was collected by filtration, and dried under reduced pressure at 50° C. to give 0.3-0.4 hydrate of compound A (compound (Ah)) as Form IX crystal (831 mg). Melting point 89-97° C.

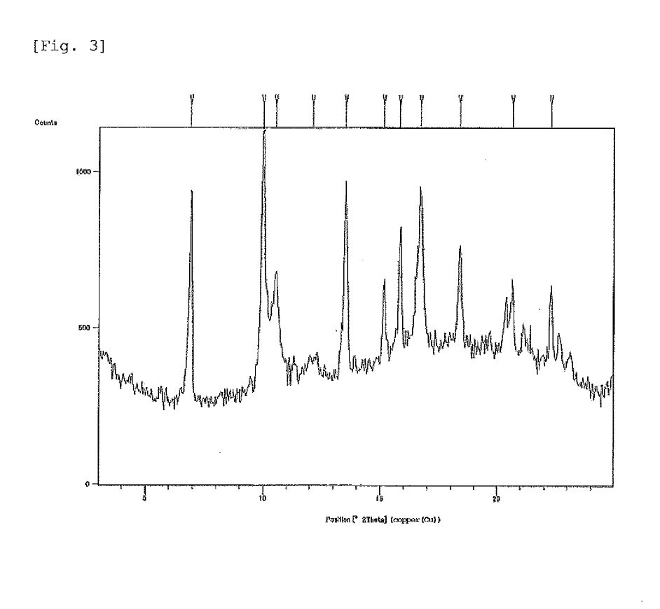

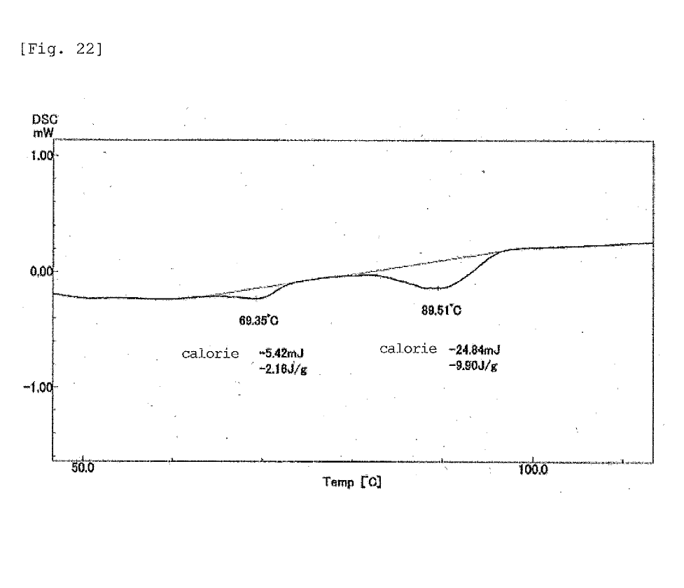

Form IX crystal was subjected to powder X-ray diffraction analysis and thermoanalysis under conditions similar to those in Example 1-1. The powder X-ray diffraction pattern and DSC curve of Form IX crystal are shown in FIG. 3 and FIG. 22, respectively.

### Example 1-4

Compound A (20 mg) obtained in Example 1 was suspended in 33 v/v % methanol water (0.15 ml), and the suspension was stood at room temperature for 5 days. The precipitated solid was collected by filtration, and dried under reduced pressure at room temperature to give 0.5 hydrate of compound A as Form II crystal (19 mg). Form II crystal was a crystal habit of Form I crystal. Melting point 89-121° C.

Form II crystal was subjected to powder X-ray diffraction analysis under conditions similar to those in Example 1-1.

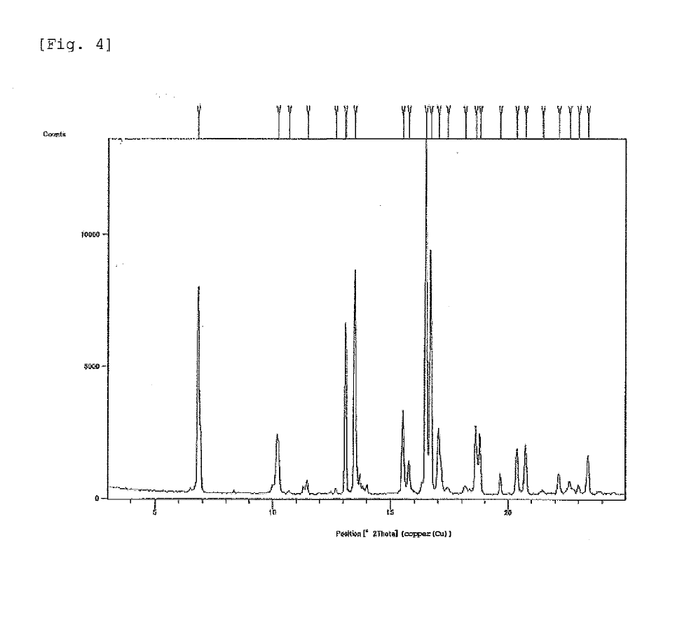

The powder X-ray diffraction pattern is shown in FIG. 4.

### Example 1-5

Compound A (50 mg) obtained in Example 1 was suspended in 30 v/v % ethanol water (0.10 ml), and the mixture was stirred at room temperature for 1 hr. The precipitated solid was collected by filtration, and dried under reduced pressure at room temperature to give ethanol solvate of compound A as Form III crystal (36 mg). Melting point 66-83° C.

Form III crystal was subjected to powder X-ray diffraction analysis under conditions similar to those in Example 1-1.

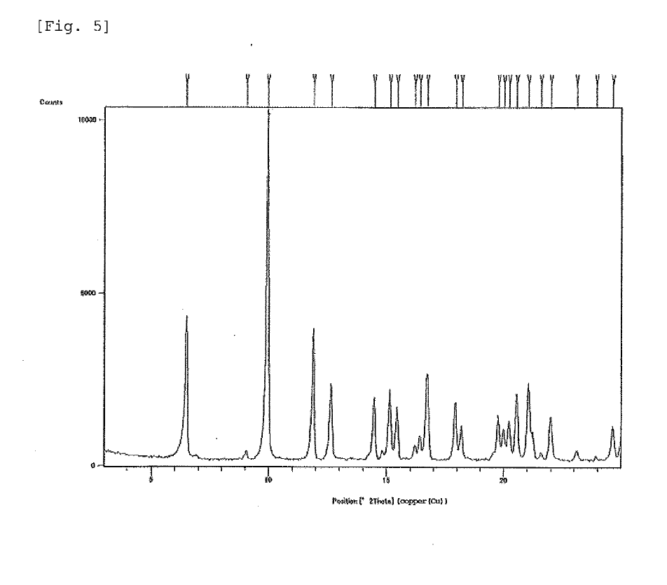

The powder X-ray diffraction pattern is shown in FIG. 5.

### Example 1-6

Compound A (1.00 g) obtained in Example 1 was suspended in 29 v/v % methanol water (7.0 ml), and the mixture was stirred at 50° C. for 45 min, and at room temperature overnight. The precipitated solid was collected by filtration to give methanol solvate of compound A as Form IV crystal (1.05 g). Melting point 89-95° C.

Form IV crystal was subjected to powder X-ray diffraction analysis under conditions similar to those in Example 1-1.

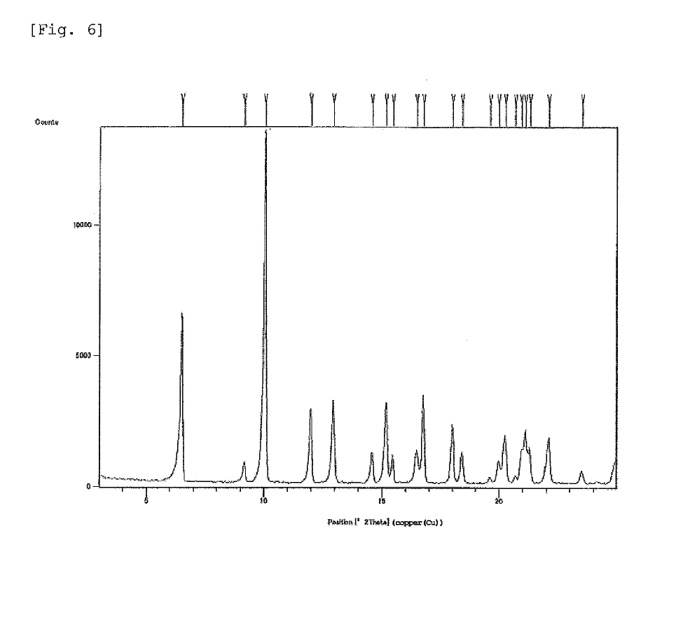

The powder X-ray diffraction pattern is shown in FIG. 6.

### Example 1-7

Compound A (50 mg) obtained in Example 1 was suspended in 30 v/v % isopropanol water (0.10 ml), and the suspension was stirred at room temperature overnight. The precipitated solid was collected by filtration, and dried under reduced pressure at room temperature to give isopropanol solvate of compound A as Form VI crystal (44 mg). Melting point 69-102° C.

Form VI crystal was subjected to powder X-ray diffraction analysis under conditions similar to those in Example 1-1.

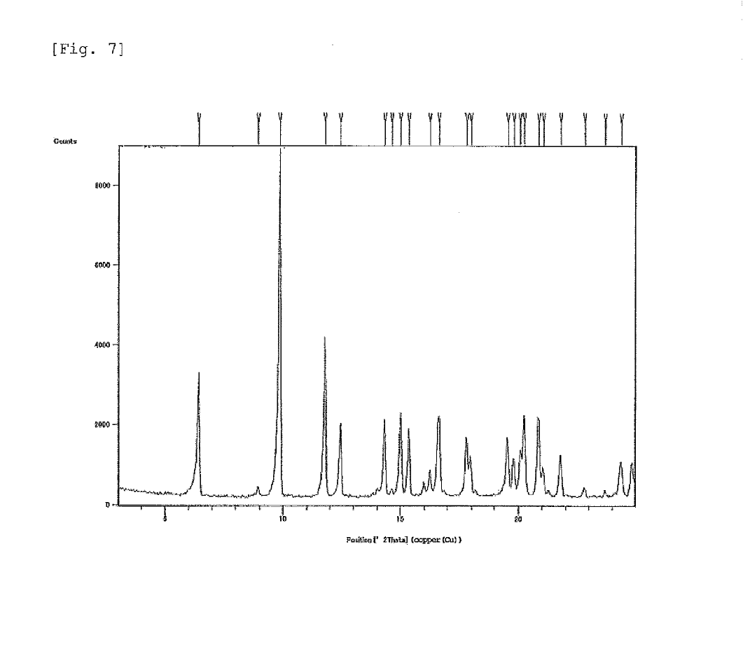

The powder X-ray diffraction pattern is shown in FIG. 7.

### Example 1-8

Compound A (50 mg) obtained in Example 1 was suspended in 30 v/v % acetone water (0.10 ml), and the suspension was stirred at room temperature for 4 days. The precipitated solid was collected by filtration, and dried under reduced pressure at room temperature to give acetone solvate of compound A as Form VII crystal (38 mg). Melting point 79-98° C.

Form VII crystal was subjected to powder X-ray diffraction analysis under conditions similar to those in Example 1-1.

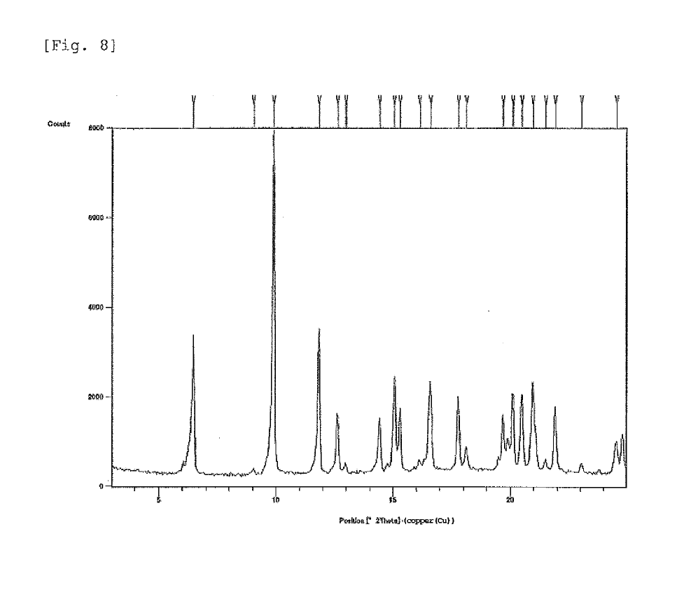

The powder X-ray diffraction pattern is shown in FIG. 8.

### Example 1-9

Compound A (50 mg) obtained in Example 1 was suspended in 30 v/v % acetic acid water (0.10 ml), and the suspension was stirred at room temperature for 1 hr. The precipitated solid was collected by filtration, and dried under reduced pressure at room temperature to give acetic acid solvate of compound A as Form VIII crystal (40 mg). Melting point 59-75° C.

Form VIII crystal was subjected to powder X-ray diffraction analysis under conditions similar to those in Example 1-1.

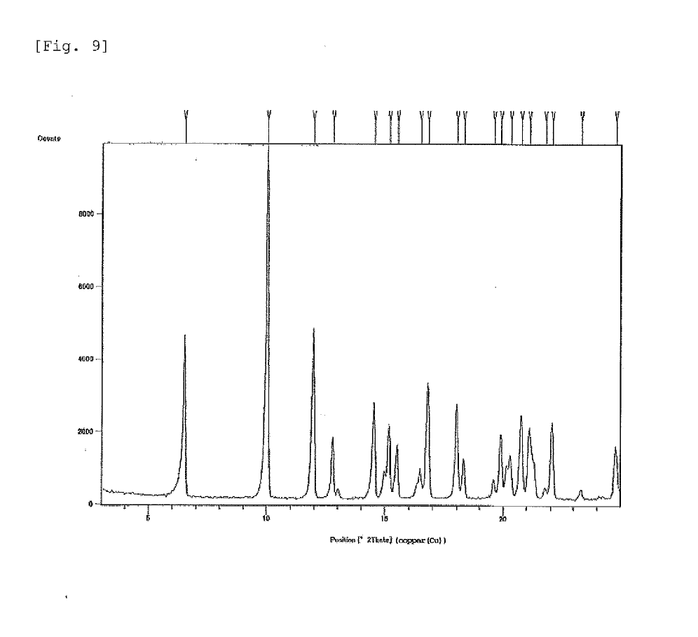

The powder X-ray diffraction pattern is shown in FIG. 9.

### Example 1-10

Form IX crystal (50 mg) of compound A obtained in Example 1-3 was suspended in toluene (0.20 ml), and the suspension was stirred at room temperature for 6 days. The precipitated solid was collected by filtration, and dried under reduced pressure at room temperature to give hydrate of compound A as Form I+X crystal (34 mg). Melting point 93-99° C.

Form I+X crystal was subjected to powder X-ray diffraction analysis under conditions similar to those in Example 1-1.

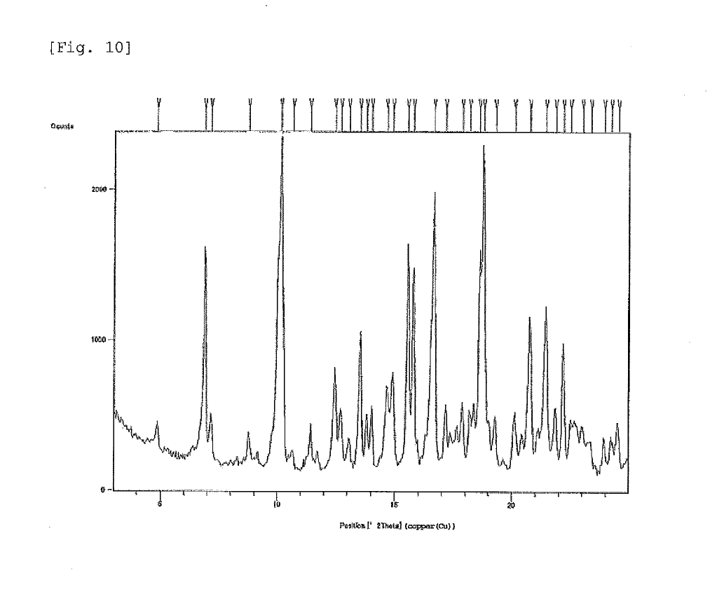

The powder X-ray diffraction pattern is shown in FIG. 10.

To obtain crystal of compound A (including solvate), about 230 crystallization conditions including the above-mentioned Example 1-1 to Example 1-10 were tested. As a result, 9 kinds of crystal polymorphisms including the above-mentioned 3 kinds of hydrates could be confirmed. All of these crystal forms could be converted to Form I crystals by a slurry method in water or an aprotic solvent, and could be obtained with good reproducibility.

### Example 2

### Synthesis of 2-hydroxymethyl-2-{4-[(9R)-9-hydroxy-2-methyl-9-(trifluoromethyl)-9H-fluoren-4-yl]-1H-pyrazol-1-yl}-propane-1,3-diol (compound B)

### Step 1

### Ethyl 2′-chloro-4′-methylbiphenyl-2-carboxylate

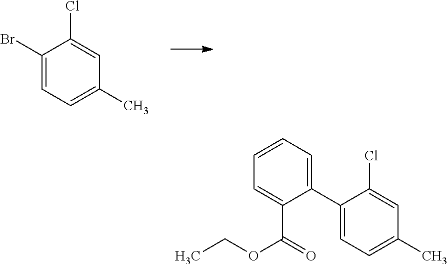

Under an argon atmosphere, 4-bromo-3-chlorotoluene (200 g), ethyl 2-(4,4,5,5-tetramethyl[1,3,2]dioxaborolan-2-yl)benzoate (376 g), toluene (1000 ml), water (1000 ml), tripotassium phosphate (412 g) and dichlorobis(triphenylphosphine)palladium(II) (14 g) were added to a reaction vessel, and the mixture was stirred at 110° C. for 2 hr. The reaction mixture was cooled to room temperature. The insoluble material was filtered off, and washed with water (500 ml) and toluene (500 ml). The filtrate was poured into a separating funnel and partitioned. The organic layer was washed twice with water (1000 ml), dried over anhydrous sodium sulfate, and filtered. The filtrate was concentrated under reduced pressure to give the title compound (337 g). The obtained residue was used for the next reaction without further purification.

¹H-NMR (400 MHz, CDCl₃) δ: 8.02-7.99 (1H, m), 7.58-7.53 (1H, m), 7.48-7.43 (1H, m), 7.28-7.23 (2H, m), 7.13-7.11 (2H, m), 4.17-4.08 (2H, m), 2.38 (3H, s), 1.06 (3H, t, J=7.1 Hz).

### Step 2

### 2′-Chloro-4′-methylbiphenyl-2-carboxylic acid

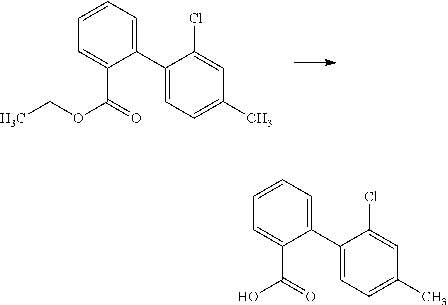 [SMILES: CCOC(=O)c1ccccc1-c1ccc(C)cc1Cl ; Cc1ccc(-c2ccccc2C(=O)O)c(Cl)c1]

To a mixture of ethyl 2′-chloro-4′-methylbiphenyl-2-carboxylate (337 g) and ethanol (728 ml) was added 4N aqueous sodium hydroxide solution (728 ml), and the mixture was stirred at 80° C. for 2 hr. The reaction mixture was cooled to room temperature, activated carbon (17 g) was added, and the mixture was stirred overnight. The activated carbon was filtered off and washed with 50 v/v % ethanol water (200 ml). The filtrate was acidified by adding acetic acid (500 ml) dropwise at room temperature. Water (414 ml) was added dropwise to the mixture at room temperature, and the mixture was stirred for 2 hr. The suspension was filtered, and the obtained solid was washed with 40 v/v % ethanol water (250 ml) and dried under reduced pressure at 80° C. to give the title compound (203 g).

¹H-NMR (400 MHz, DMSO-D₆) δ: 12.60 (1H, br s), 7.93-7.89 (1H, m), 7.64-7.58 (1H, m), 7.53-7.47 (1H, m), 7.32-7.29 (1H, m), 7.25-7.21 (1H, m), 7.20-7.13 (2H, m), 2.34 (3H, s).

### Step 3

### 4-Chloro-2-methyl-9H-fluoren-9-one

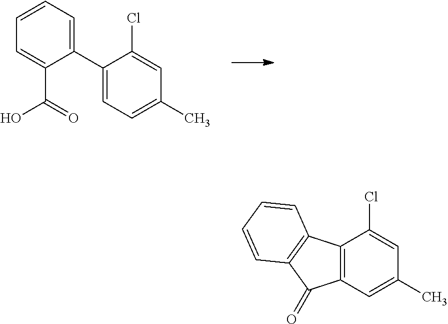 [SMILES: Cc1ccc(-c2ccccc2C(=O)O)c(Cl)c1 ; Cc1cc(Cl)c2c(c1)C(=O)c1ccccc1-2]

To a mixture of phosphorus pentoxide (V) (150 g) and methanesulfonic acid (1500 ml) was added 2′-chloro-4′-methylbiphenyl-2-carboxylic acid (153 g), and the mixture was stirred at 80° C. for 2 hr. The reaction mixture was cooled to 0° C. Water (1500 ml) was added dropwise while keeping the temperature of the reaction mixture at 90° C. or below, and the mixture was further stirred at room temperature for 2 hr. The suspension was filtered, and the obtained solid was washed with water (1000 ml). The solid was suspended in 50 v/v % ethanol water (1500 ml), slurry-washed (suspension stirred) at room temperature for 2 hr, and filtered. The obtained solid was air-dried for 1 hr and dried under reduced pressure at 80° C. to give the title compound (140.12 g).

¹H-NMR (400 MHz, DMSO-D₆) δ: 8.10-8.07 (1H, m), 7.69-7.64 (2H, m), 7.49-7.41 (3H, m), 2.36 (3H, s).

### Step 4

### [4-Chloro-2-methyl-9-(trifluoromethyl)-9H-fluoren-9-yloxy]acetic acid

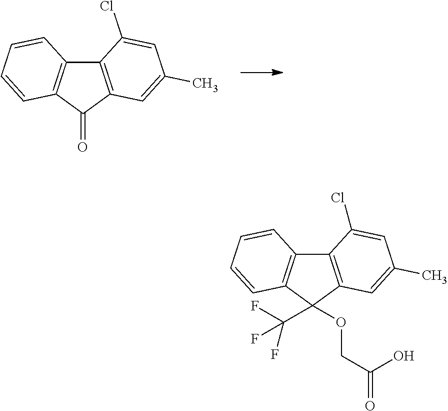 [SMILES: Cc1cc(Cl)c2c(c1)C(=O)c1ccccc1-2 ; Cc1cc(Cl)c2c(c1)C(OCC(=O)O)(C(F)(F)F)c1ccccc1-2]

Under an argon flow, to a mixture of 4-chloro-2-methyl-9H-fluoren-9-one (100 g) and dimethylformamide (500 ml) was added potassium carbonate (18 g). To the mixture was added dropwise trimethyl(trifluoromethyl)silane (78 ml) over 80 min, and the mixture was further stirred at room temperature for 1 hr. Cesium fluoride (87 g) was added to the reaction mixture at room temperature, then ethyl bromoacetate (63 ml) was added dropwise over 15 min, and the mixture was further stirred at room temperature for 4 hr. Water (500 ml) was added to the reaction mixture, and the aqueous layer was extracted twice with toluene (500 ml). The combined organic layer was washed with water (500 ml) and saturated brine (500 ml), dried over anhydrous sodium sulfate, and filtered. The filtrate was concentrated under reduced pressure. Ethanol (220 ml) and 2N aqueous sodium hydroxide solution (440 ml) were added to the obtained residue, and the mixture was stirred at 80° C. for 1 hr. The reaction mixture was cooled to room temperature, activated carbon (15 g) was added, and the mixture was stirred at room temperature overnight. The activated carbon was filtered off, and washed with 33 v/v % ethanol water (120 ml). The filtrate was acidified by adding acetic acid (151 ml) dropwise and the mixture was stirred at room temperature overnight. The suspension was filtered, and the obtained solid was washed with 33 v/v % ethanol water (150 ml) and dried under reduced pressure at 80° C. to give the title compound (136.40 g).

¹H-NMR (400 MHz, DMSO-D₆) δ: 12.76 (1H, br s), 8.26 (1H, d, J=7.7 Hz), 7.69-7.62 (2H, m), 7.53-7.45 (3H, m), 3.50 (1H, d, J=15.5 Hz), 3.43 (1H, d, J=15.5 Hz), 2.41 (3H, s).

### Step 5

### (1R)-1-Phenylethylamine salt of [(9R)-4-chloro-2-methyl-9-(trifluoromethyl)-9H-fluoren-9-yloxy]acetic acid

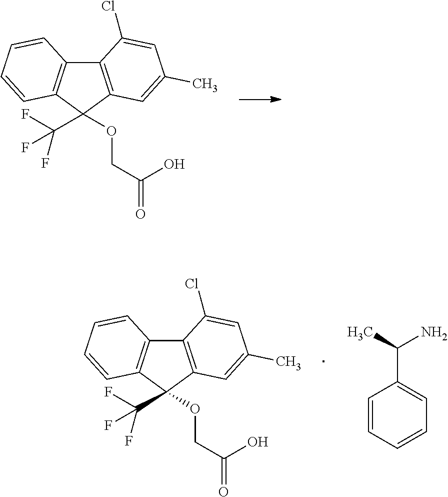

### Step 5-1

### Preparation of Seed Crystal

To a mixture of [4-chloro-2-methyl-9-(trifluoromethyl)-9H-fluoren-9-yloxy]acetic acid (0.400 g) and isopropyl ether (16 ml) was added (1R)-1-phenylethylamine (0.058 ml). The mixture was stirred at room temperature for 1 hr 40 min. The suspension was filtered, and the residue was dried under reduced pressure to give a solid (0.240 g). The solid (0.210 g) was suspended in ethyl acetate (4.2 mL), and the suspension was stirred at room temperature for 1 hr. The suspension was filtered, and the residue was dried under reduced pressure to give a solid (0.178 g). The solid (0.170 g) was resuspended in ethyl acetate (3.4 mL), and the suspension was stirred at 50° C. for 1 hr. The suspension was filtered, and the obtained solid was dried under reduced pressure to give the title compound (0.137 g). The solid was subjected to inducing method A for optical purity determination and analyzed under HPLC analysis condition 1. It contained a large amount of (R)-form carboxylic acid derivatives, and the optical purity was 96.7% e.e.

(S)-form carboxylic acid derivative (retention time 20.19 min)

(R)-form carboxylic acid derivative (retention time 21.41 min)

¹H-NMR (400 MHz, DMSO-D₆) δ: 8.24 (1H, d, J=7.7 Hz), 7.70 (3H, br s), 7.67-7.57 (2H, m), 7.51-7.40 (5H, m), 7.39-7.32 (2H, m), 7.32-7.26 (1H, m), 4.22 (1H, q, J=6.8 Hz), 3.12 (1H, d, J=14.0 Hz), 3.08 (1H, d, J=14.0 Hz), 2.40 (3H, s), 1.39 (3H, d, J=6.8 Hz).

### Step 5-2

To a mixture of [4-chloro-2-methyl-9-(trifluoromethyl)-9H-fluoren-9-yloxy]acetic acid (191.60 g) and methyl isobutyl ketone (575 ml) was added (1R)-1-phenylethylamine (34.81 ml). To the mixture was added a seed crystal, and the mixture was stirred at 50° C. for 3 days. The suspension was filtered, and the obtained solid was washed with methyl isobutyl ketone (192 ml), and dried under reduced pressure to give the title compound (71.10 g). In the same manner as in step 5-1, the solid was subjected to inducing method A for optical purity determination and analyzed under HPLC analysis condition 1. It contained a large amount of (R)-form carboxylic acid derivatives, and the optical purity was 95.0% e.e.

### (Absolute Configuration)

The absolute configuration of [4-chloro-2-methyl-9-(trifluoromethyl)-9H-fluoren-9-yloxy]acetic acid was determined to be (R) by single crystal X-ray structure analysis.

### Step 6

### [(9R)-4-Chloro-2-methyl-9-(trifluoromethyl)-9H-fluoren-9-yloxy]acetic acid

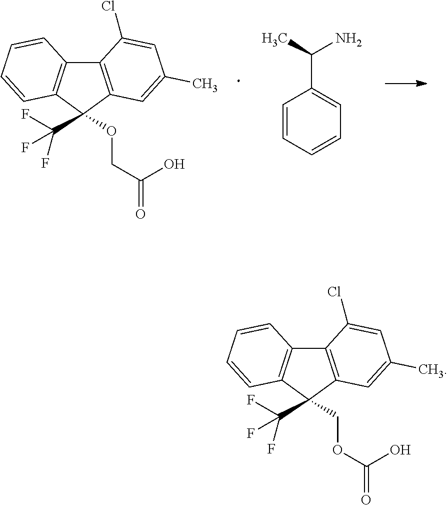

To a mixture of (1R)-1-phenylethylamine salt of [(9R)-4-chloro-2-methyl-9-(trifluoromethyl)-9H-fluoren-9-yloxy]acetic acid (159.16 g) and ethyl acetate (796 ml) was added 2N hydrochloric acid (318 ml), and the mixture was stirred at room temperature for 2 hr. The mixture was poured into a separating funnel, and partitioned. The organic layer was washed twice with water (600 ml) and once with saturated brine (300 ml), dried over anhydrous sodium sulfate, and filtered. The filtrate was concentrated under reduced pressure, hexane was added to the obtained residue, and the mixture was slurry-washed (suspension stirred) at room temperature for 1 hr. The suspension was filtered and washed with hexane. The obtained solid was dried under reduced pressure to give the title compound (112.33 g).

¹H-NMR (400 MHz, DMSO-D₆) δ: 12.75 (1H, br s), 8.26 (1H, d, J=7.7 Hz), 7.71-7.62 (2H, m), 7.54-7.45 (3H, m), 3.50 (1H, d, J=15.5 Hz), 3.43 (1H, d, J=15.5 Hz), 2.41 (3H, s).

### Step 7

### (9R)-4-Chloro-2-methyl-9-(trifluoromethyl)-9H-fluoren-9-ol

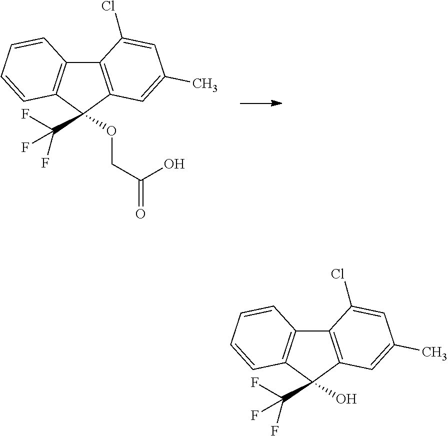 [SMILES: Cc1cc(Cl)c2c(c1)[C@@](OCC(=O)O)(C(F)(F)F)c1ccccc1-2 ; Cc1cc(Cl)c2c(c1)[C@@](O)(C(F)(F)F)c1ccccc1-2]

To a mixture of [(9R)-4-chloro-2-methyl-9-(trifluoromethyl)-9H-fluoren-9-yloxy]acetic acid (30 g) and dimethylformamide (90 ml) was added triethylamine (14.1 and the mixture was stirred at 0° C. A solution of diphenylphosphoryl azide (20.0 ml) in dimethylformamide (60 ml) was added dropwise thereto over 20 min, and the mixture was further stirred at 0° C. for 2 hr. t-Butyl alcohol (75 ml) was added to the reaction mixture, and the mixture was stirred at 100° C. for 1 hr. The reaction mixture was ice-cooled, 2N hydrochloric acid (300 ml) was added, and the mixture was stirred at room temperature overnight. Water (100 ml) was added to the mixture, the mixture was poured into a separating funnel, and the aqueous layer was extracted twice with toluene (300 ml, 200 ml). The combined organic layer was successively washed twice with water (200 ml), twice with 1N aqueous sodium hydroxide solution (150 ml) and once with saturated brine (150 ml). To the obtained organic layer were added anhydrous sodium sulfate and silica gel (6 g), and the mixture was stirred at room temperature. The insoluble material was filtered off and washed with toluene (500 ml). The filtrate was concentrated under reduced pressure to give the title compound (28.58 g). Specific optical rotation [α]_{D}=+22.50° (20° C., c=1.00, methanol).

¹H-NMR (400 MHz, CDCl₃) δ: 8.27 (1H, d, J=7.7 Hz), 7.70-7.69 (1H, m), 7.52-7.47 (1H, m), 7.44-7.42 (1H, m), 7.40-7.35 (1H, m), 7.25 (1H, s), 2.82 (1H, br s), 2.41 (3H, s).

### Step 8

### t-Butyl[4-(4,4,5,5-tetramethyl[1,3,2]dioxaborolan-2-yl)-1H-pyrazol-1-yl]acetate

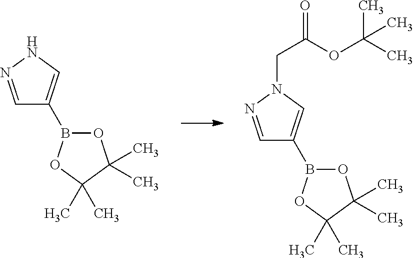 [SMILES: CC1(C)OB(c2cn[nH]c2)OC1(C)C ; CC(C)(C)OC(=O)Cn1cc(B2OC(C)(C)C(C)(C)O2)cn1]

4,4,5,5-Tetramethyl-2-(1H-pyrazol-4-yl)[1,3,2]dioxaborolane (10 g), N,N-dimethylacetamide (100 ml), potassium carbonate (17.8 g) and t-butyl bromoacetate (9.9 mL) were mixed, and the mixture was stirred at room temperature for 4 hr. The reaction mixture was filtered through celite. Water and diethylether were added to the filtrate, and the mixture was poured into a separating funnel and partitioned. The aqueous layer was extracted again with diethyl ether, and combined with the organic layer. The obtained organic layer was washed three times with water, once with saturated brine, and dried over anhydrous sodium sulfate. The insoluble material was filtered off, and the filtrate was concentrated under reduced pressure. To the obtained residue was added hexane (50 ml) and the mixture was slurry washed (suspension stirred). The suspension was filtered, and the obtained solid was washed with hexane, and dried under reduced pressure to give the title compound (12.23 g).

¹H-NMR (400 MHz, DMSO-D₆) δ: 7.92 (1H, d, J=0.7 Hz), 7.59 (1H, d, J=0.5 Hz), 4.95 (2H, s), 1.42 (9H, s), 1.25 (12H, s).

### Step 9

### t-Butyl {4-[(9R)-9-hydroxy-2-methyl-9-(trifluoromethyl)-9H-fluoren-4-yl]-1H-pyrazol-1-yl}acetate

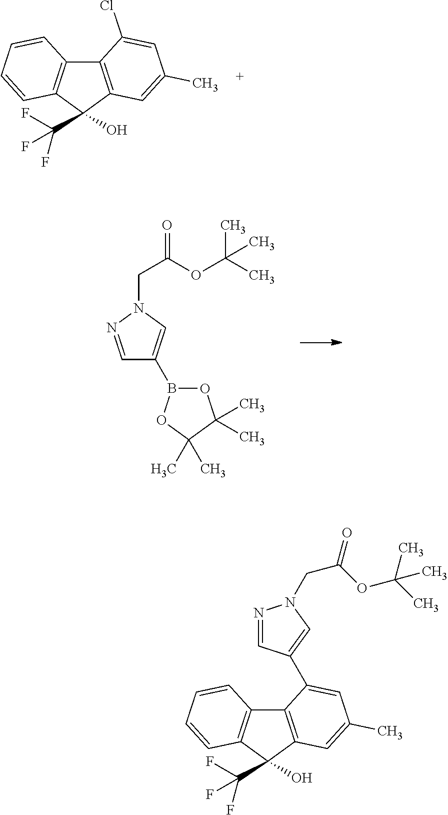 [SMILES: Cc1cc(Cl)c2c(c1)[C@@](O)(C(F)(F)F)c1ccccc1-2 ; CC(C)(C)OC(=O)Cn1cc(B2OC(C)(C)C(C)(C)O2)cn1 ; Cc1cc(-c2cnn(CC(=O)OC(C)(C)C)c2)c2c(c1)[C@@](O)(C(F)(F)F)c1ccccc1-2]

To a suspension of t-butyl[4-(4,4,5,5-tetramethyl[1,3,2]dioxaborolan-2-yl)-1H-pyrazol-1-yl]acetate (24.8 g), (9R)-4-chloro-2-methyl-9-(trifluoromethyl)-9H-fluoren-9-ol (20.0 g) and sodium hydrogen carbonate (11.3 g) in toluene/water (200 ml/60 ml) were added palladium acetate (750 mg) and 2-dicyclohexylphosphino-2′,6′-dimethoxybiphenyl (2.75 g) at room temperature, and the mixture was stirred at 110° C. for 2 hr. The reaction mixture was cooled to room temperature, water (80 ml) and activated carbon (2.0 g) were added, and the mixture was stirred for 1 hr. The mixture was filtered through celite, and the solid was washed with tetrahydrofuran (100 ml). The filtrate was partitioned, and the aqueous layer was extracted with ethyl acetate (100 ml). The combined organic layer was successively washed twice with water (100 ml) and once with saturated brine (100 ml). Anhydrous sodium sulfate and silica gel (40 g) were added, and the mixture was stirred overnight. The mixture was filtered, and the filtrate was concentrated under reduced pressure. The obtained residue was suspended in a mixed solution of hexane and ethyl acetate (hexane:ethyl acetate mixing ratio 2:1, 120 ml), and the solid was collected by filtration to give the title compound (17.8 g).

¹H-NMR (400 MHz, CDCl₃) δ: 7.68-7.64 (1H, m), 7.65 (1H, d, J=0.7 Hz), 7.60 (1H, d, J=0.7 Hz), 7.51-7.49 (1H, m), 7.40-7.37 (1H, m), 7.28-7.23 (2H, m), 7.14-7.13 (1H, m), 4.93 (1H, d, J=17.4 Hz), 4.88 (1H, d, J=17.4 Hz), 4.80 (1H, s), 2.42 (3H, s), 1.52 (9H, s).

### Step 10

### 3-Hydroxy-2-hydroxymethyl-2-{4-[(9R)-9-hydroxy-2-methyl-9-(trifluoromethyl)-9H-fluoren-4-yl]-1H-pyrazol-1-yl}propionic acid

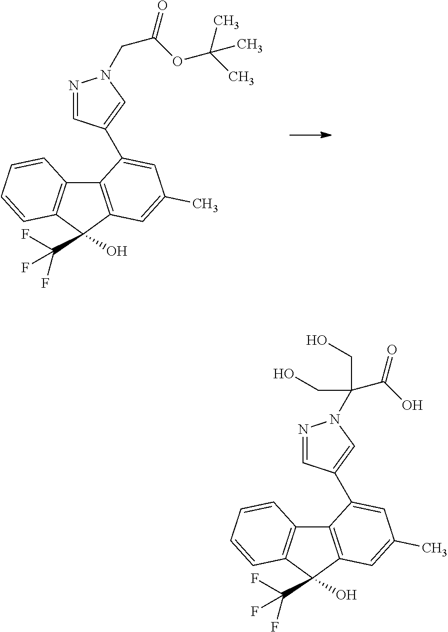 [SMILES: Cc1cc(-c2cnn(CC(=O)OC(C)(C)C)c2)c2c(c1)[C@@](O)(C(F)(F)F)c1ccccc1-2 ; Cc1cc(-c2cnn(C(CO)(CO)C(=O)O)c2)c2c(c1)[C@@](O)(C(F)(F)F)c1ccccc1-2]

To a solution of t-butyl {4-[(9R)-9-hydroxy-2-methyl-9-(trifluoromethyl)-9H-fluoren-4-yl]-1H-pyrazol-1-yl}acetate (17.8 g) and paraformaldehyde (12.0 g) in dimethylformamide (60 ml) was added a 1M tetrahydrofuran solution (120 ml) of tetrabutylammonium fluoride at room temperature, and the mixture was stirred at 95° C. for 3 hr. 1N Hydrochloric acid (180 ml) and water (90 ml) were added to the reaction mixture, and the mixture was extracted with ethyl acetate (180 ml). The separated aqueous layer was extracted twice with ethyl acetate (90 ml). The combined organic layer was successively washed once with 1N hydrochloric acid (90 ml), twice with water (90 ml), and once with saturated brine (90 ml). The organic layer was dried over anhydrous sodium sulfate, and filtered. The filtrate was concentrated under reduced pressure to give the title compound (15.5 g).

¹H-NMR (400 MHz, DMSO-D₆) δ: 13.10 (1H, br s), 8.03 (1H, s), 7.65 (1H, s), 7.63-7.59 (1H, m), 7.44-7.40 (2H, m), 7.31-7.27 (1H, m), 7.25-7.20 (1H, m), 7.15 (2H, s), 5.14 (2H, br s), 4.21-4.09 (4H, m), 2.39 (3H, s).

### Step 11

### 2-Hydroxymethyl-2-{4-[(9R)-9-hydroxy-2-methyl-9-(trifluoromethyl)-9H-fluoren-4-yl]-1H-pyrazol-1-yl}-propane-1,3-diol (compound B)

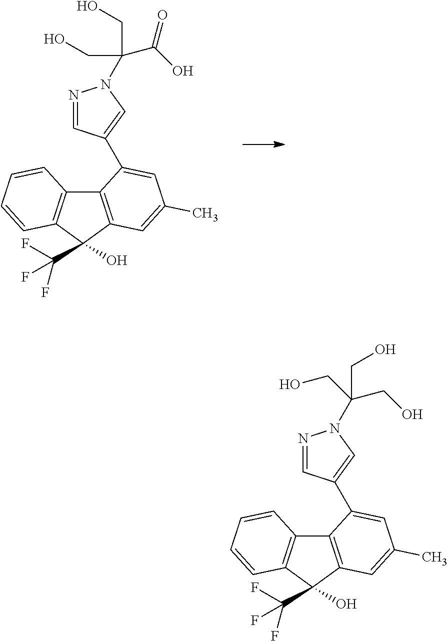 [SMILES: Cc1cc(-c2cnn(C(CO)(CO)C(=O)O)c2)c2c(c1)[C@@](O)(C(F)(F)F)c1ccccc1-2 ; Cc1cc(-c2cnn(C(CO)(CO)CO)c2)c2c(c1)[C@@](O)(C(F)(F)F)c1ccccc1-2]

To a solution of 3-hydroxy-2-hydroxymethyl-2-{4-[(9R)-9-hydroxy-2-methyl-9-(trifluoromethyl)-9H-fluoren-4-yl)-1H-pyrazol-1-yl}propionic acid (15.5 g) in tetrahydrofuran (31 ml) was added dropwise a 1.09M tetrahydrofuran solution (127 ml) of borane-tetrahydrofuran complex at room temperature, and the mixture was stirred for 5 hr. Ethanol (15 ml) was added dropwise to the reaction mixture at room temperature, and the mixture was stirred at 75° C. for 1 hr. To the mixture were added water (90 ml) and saturated aqueous sodium hydrogen carbonate solution (150 ml), and the mixture was extracted twice with ethyl acetate (150 ml, 75 ml). The combined organic layer was successively washed once with saturated aqueous sodium hydrogen carbonate solution (75 ml), twice with water (75 ml), and once with saturated brine (75 ml), dried over anhydrous sodium sulfate and concentrated under reduced pressure. To a solution of the obtained residue in ethanol (45 ml) was added sodium borohydride (1.3 g) at room temperature, and the mixture was stirred for 1 hr. To the reaction mixture was added 1N hydrochloric acid (150 ml), and the mixture was extracted with ethyl acetate (150 ml). The separated aqueous layer was extracted again with ethyl acetate (75 ml). The combined organic layer was successively washed with water (75 ml), saturated aqueous sodium hydrogen carbonate solution (75 ml), water (75 ml), and saturated brine (75 ml). The obtained organic layer was dried over anhydrous sodium sulfate, and filtered. The filtrate was concentrated under reduced pressure. The obtained residue was purified by silica gel column chromatography (a mixture of chloroform and methanol was used as an elution solvent; the mixture was first eluted with chloroform: methanol at a mixing ratio 20:1, and further at a mixing ratio 10:1) to give the title compound (12.6 g). Specific optical rotation [α]_{D}=+65.6° (25° C., c=1.008, methanol).

¹H-NMR (400 MHz, DMSO-D₆) δ: 7.96 (1H, d, J=0.7 Hz), 7.63-7.59 (1H, m), 7.62 (1H, d, J=0.7 Hz), 7.46-7.40 (2H, m), 7.31-7.21 (2H, m), 7.14 (2H, s), 4.82 (3H, t, J 5.6 Hz), 3.91 (6H, d, J=5.6 Hz), 2.39 (3H, s).

### Example 2-1

Compound B (30.2 g) obtained in Example 2 was suspended in 30 v/v % methanol water (200 ml), and the suspension was stirred at 50° C. for 4 hr, and at room temperature for 4 days. The precipitated solid was collected by filtration, and dried under reduced pressure at room temperature to give 2 hydrate of compound B (compound (Bh)) as Form IVb crystal (28.7 g).

Melting point 96-104° C.

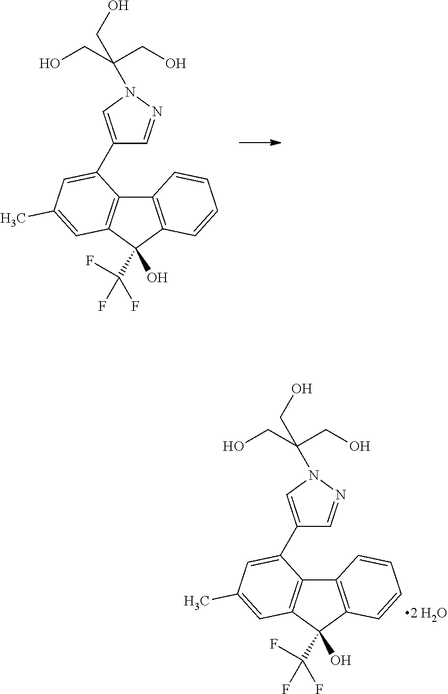 [SMILES: Cc1cc(-c2cnn(C(CO)(CO)CO)c2)c2c(c1)[C@@](O)(C(F)(F)F)c1ccccc1-2 ; Cc1cc(-c2cnn(C(CO)(CO)CO)c2)c2c(c1)[C@@](O)(C(F)(F)F)c1ccccc1-2]

### (Powder X-Ray Diffraction Analysis)

Form IVb crystal of compound (Bh) was subjected to powder X-ray diffraction analysis under conditions similar to those in Example 1-1.

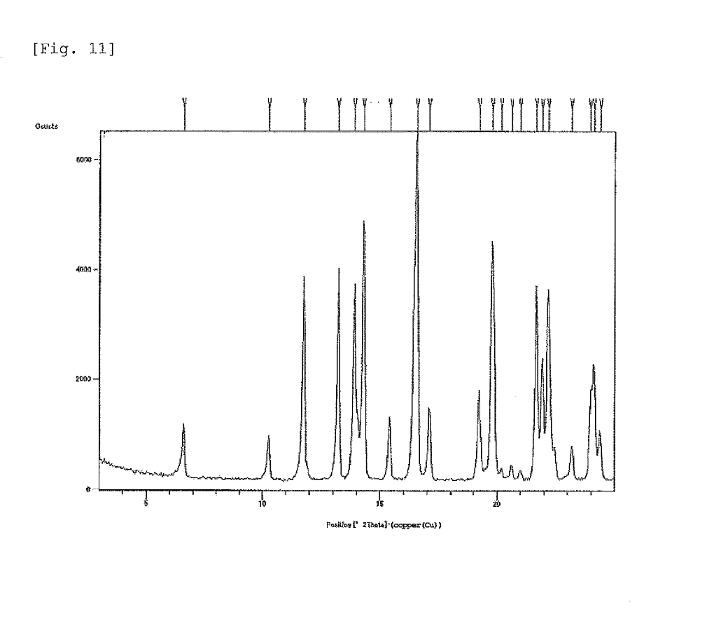

The powder X-ray diffraction pattern is shown in FIG. 11, and the relative intensity, wherein the peak intensity at diffraction angle 2θ of 16.6° is 100, is shown in Table 5.

TABLE 5

| | relative | NET |
|---|---|---|
| | intensity | intensity |
| Pos. [°2Th.] | [%] | [cts] |
| 6.5961 | 15.20 | 962.89 |
| 10.2647 | 13.13 | 831.67 |
| 11.7561 | 61.24 | 3878.21 |
| 13.2157 | 61.19 | 3875.41 |
| 13.8990 | 56.48 | 3576.72 |
| 14.2972 | 76.28 | 4830.78 |
| 15.4027 | 18.18 | 1151.63 |
| 16.5556 | 100.00 | 6333.25 |
| 17.1010 | 20.99 | 1329.07 |
| 19.2254 | 25.81 | 1634.70 |
| 19.8022 | 69.77 | 4418.89 |
| 20.1950 | 3.38 | 214.11 |
| 20.6126 | 4.13 | 261.78 |
| 20.9929 | 2.67 | 169.00 |
| 21.6665 | 55.90 | 3540.60 |
| 21.9230 | 35.28 | 2234.41 |
| 22.1797 | 56.08 | 3551.80 |
| 23.1860 | 9.82 | 621.92 |
| 23.9516 | 23.38 | 1480.48 |
| 24.1124 | 32.65 | 2067.75 |
| 24.3616 | 13.51 | 855.65 |

### (Powder X-Ray Diffraction Analysis and Differential Scanning Calorimetry)

Form IVb crystal (2-3 mg) was measured using an apparatus for simultaneous measurements of powder X-ray diffraction and DSC thermogram (XRD-DSC) (XRD:RINT-2100; DSC:DSC8230, manufactured by Rigaku Corporation) under a dried nitrogen atmosphere at a temperature rise rate of 2° C./min (sealed aluminum pan). The obtained DSC curve is shown in FIG. 21. The enthalpy of the endothermic peak on the DSC curve was about 124.3 J/g, and the extrapolated onset temperature was 62.3±5° C.

### (Differential Heat/Thermogravimetry Simultaneous Measurement)

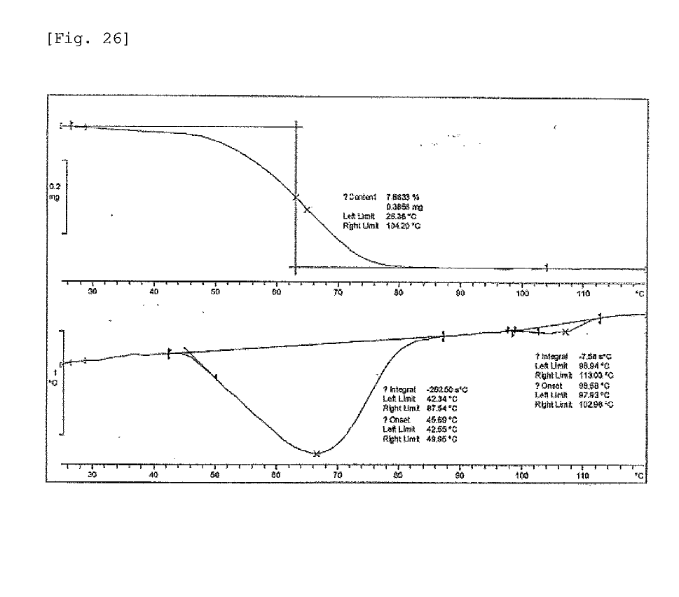

Form IVb crystal (about 5 mg) was placed in an open aluminum pan, and measured using TG-DTA measuring apparatus (TGA/SDTA851^{e}/SF, manufactured by Mettler Toledo International Inc.) under a dried nitrogen atmosphere at a temperature rise rate of 5° C./min. The measurement results are shown in FIG. 26.

According to FIG. 26, a weight loss of 7.68% was found by heating, and the decrease ratio thereof was well consistent with the theoretical water content of 7.66% of compound (Bh).

### (Elemental Analysis Measurement)

Furthermore, Form IVb crystal was subjected to elemental analysis under conditions similar to those in Example 1-1. The results of the elemental analysis of Form IVb crystal were well consistent with the calculated values of compound (Bh), as shown below.

Calculated: C, 56.17; H, 5.36; N, 5.95 (calculated as 2 hydrate);

Found: C, 56.19; H, 5.37; N, 5.97.

In addition, Form IVb crystal was subjected to water adsorption and desorption test under conditions similar to those in Example 1-1.

As a result, when pre-drying was performed (under dried nitrogen stream, 60° C.), the weight decreased by 7.75%, and the decrease ratio thereof was well consistent with the theoretical water content of 7.66% of compound (Bh). Therefore, it was assumed that pre-drying completely evaporated crystal water.

On the other hand, it is considered that 2 equivalents of water were reabsorbed by humidification, since humidification after drying up to relative humidity 50% resulted in a weight increase of 7.93%.

When pre-drying was not performed, remarkable moisture absorption was not found even under relative humidity 95%, and the rate of moisture absorption under relative humidity 75% was 0.305% to 0.354% and less than 3%. The results confirm that Form IVb crystal of compound (Bh) does not have hygroscopicity.

### Example 2-2

Compound B (200 mg) obtained in Example 2 was suspended in chloroform (4.0 ml), and the suspension was stirred at room temperature overnight. The precipitated solid was collected by filtration, and dried under reduced pressure at room temperature to give compound B as Form Ib crystal (227 mg).

Melting point 88-111° C.

Form Ib crystal was subjected to powder X-ray diffraction analysis under conditions similar to those in Example 1-1.

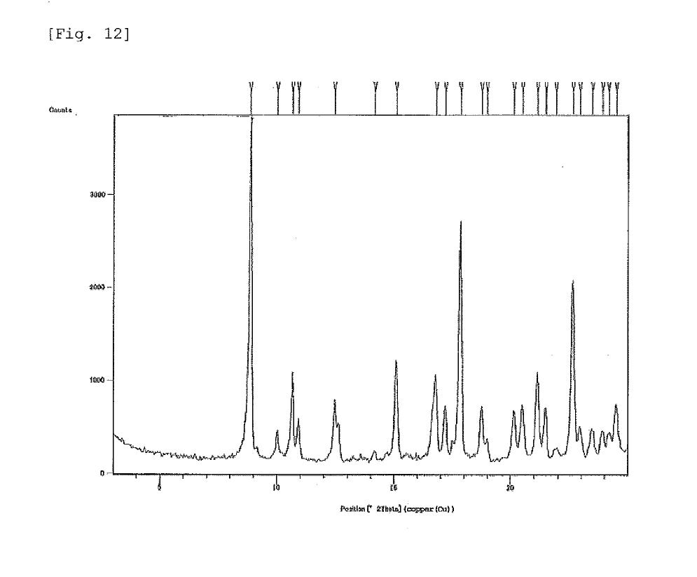

The powder X-ray diffraction pattern is shown in FIG. 12.

### Example 2-3

Compound B (20 mg) obtained in Example 2 was suspended in diethyl ether (0.20 ml), and the suspension was stirred at room temperature overnight. The precipitated solid was collected by filtration, and dried under reduced pressure at room temperature to give compound B as Form IIb crystal (13 mg).

Form IIb crystal was subjected to powder X-ray diffraction analysis under conditions similar to those in Example 1-1.

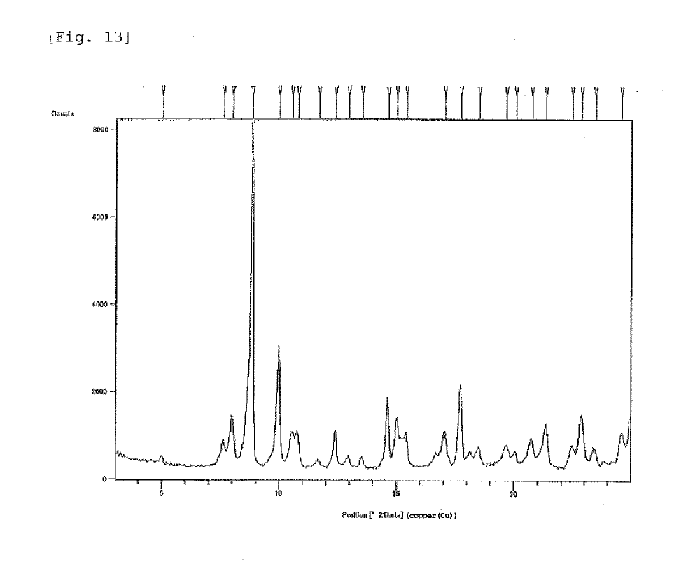

The powder X-ray diffraction pattern is shown in FIG. 13.

### Example 2-4

Compound B (20 mg) obtained in Example 2 was suspended in 1,2-dichloroethane (0.10 ml), and the suspension was stirred at room temperature overnight. The precipitated solid was collected by filtration, and dried under reduced pressure at room temperature to give compound B as Form IIIb crystal (23 mg).

Form IIIb crystal was subjected to powder X-ray diffraction analysis under conditions similar to those in Example 1-1.

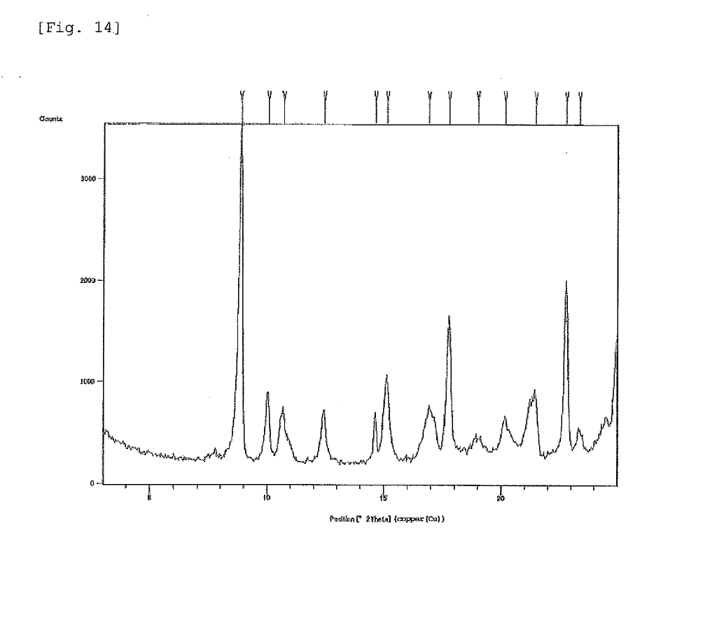

The powder X-ray diffraction pattern is shown in FIG. 14.

### Example 2-5

Form IVb crystal (50 mg) of compound (Bh) obtained in Example 2-1 was suspended in propionitrile (0.050 ml), and the suspension was stirred at room temperature for 4 days. The precipitated solid was collected by filtration, and dried under reduced pressure at room temperature to give compound B as Form Vb+IVb crystal (9 mg).

Form Vb+IVb crystal was subjected to powder X-ray diffraction analysis under conditions similar to those in Example 1-1.

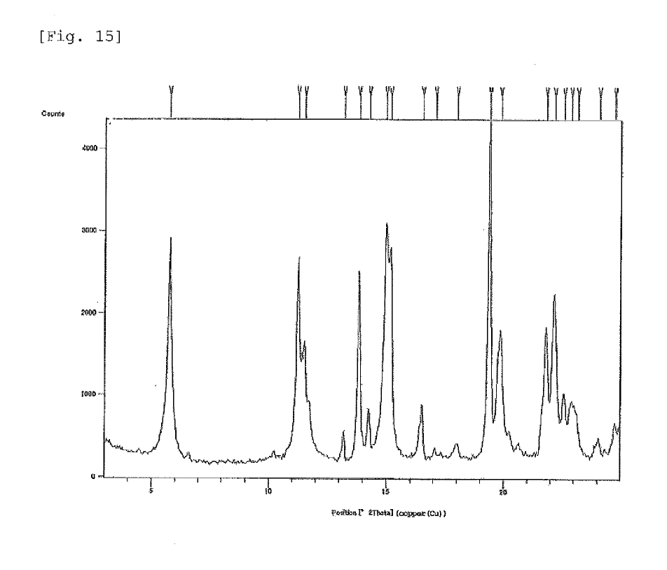

The powder X-ray diffraction pattern is shown in FIG. 15.

### Example 2-6

Compound B (200 mg) obtained in Example 3 was suspended in propionitrile (0.20 ml), Form Vb+IVb crystal of compound B obtained in Example 2-5 was inoculated, and the mixture was stood at room temperature for 30 min. The precipitated solid was collected by filtration, and dried under reduced pressure at room temperature to give compound B as Form VIb crystal (201 mg).

Form VIb crystal was subjected to powder X-ray diffraction analysis under conditions similar to those in Example 1-1.

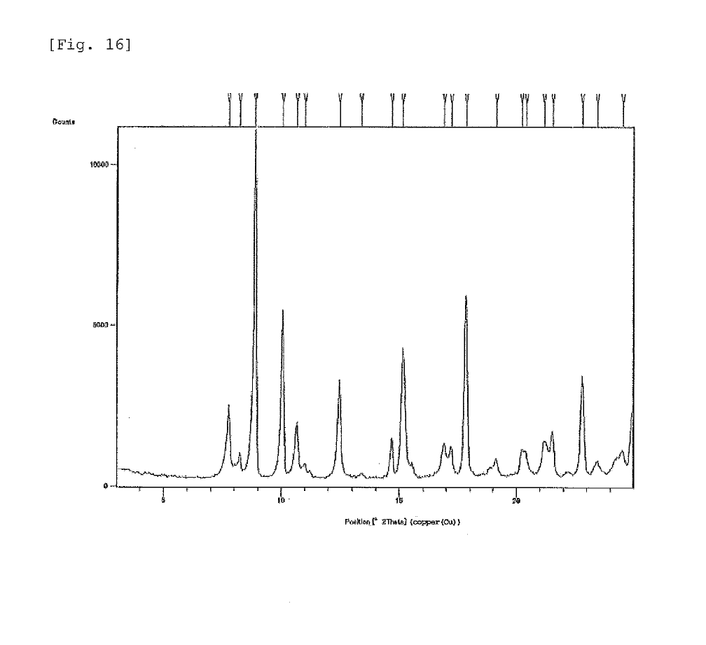

The powder X-ray diffraction pattern is shown in FIG. 16.

### Example 2-7

Compound B (200 mg) obtained in Example 2 was suspended in trimethylacetonitrile (0.20 ml), Form Vb+IVb crystal of compound B obtained in Example 2-5 was inoculated, and the mixture was stood at room temperature for 30 min. The precipitated solid was collected by filtration, and dried under reduced pressure at room temperature to give compound B as Form VIIb crystal (215 mg).

Form VIIb crystal was subjected to powder X-ray diffraction analysis under conditions similar to those in Example 1-1.

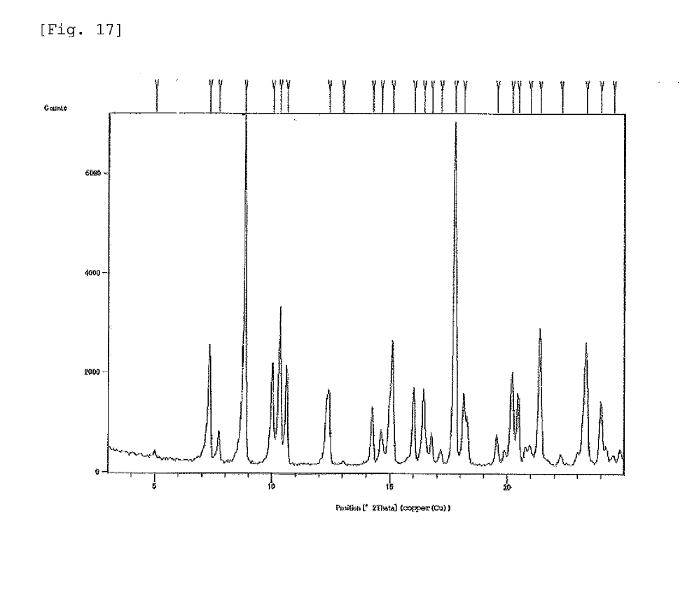

The powder X-ray diffraction pattern is shown in FIG. 17.

### Example 2-8

Compound B (50 mg) obtained in Example 2 was suspended in 2-butanol (0.050 ml), Form Ib crystal of compound B obtained in Example 2-2 was inoculated, and the mixture was stirred at room temperature for 7 days. The precipitated solid was collected by filtration, and dried under reduced pressure at room temperature to give compound B as Form VIIIb crystal (16 mg).

Form VIIIb crystal was subjected to powder X-ray diffraction analysis under conditions similar to those in Example 1-1.

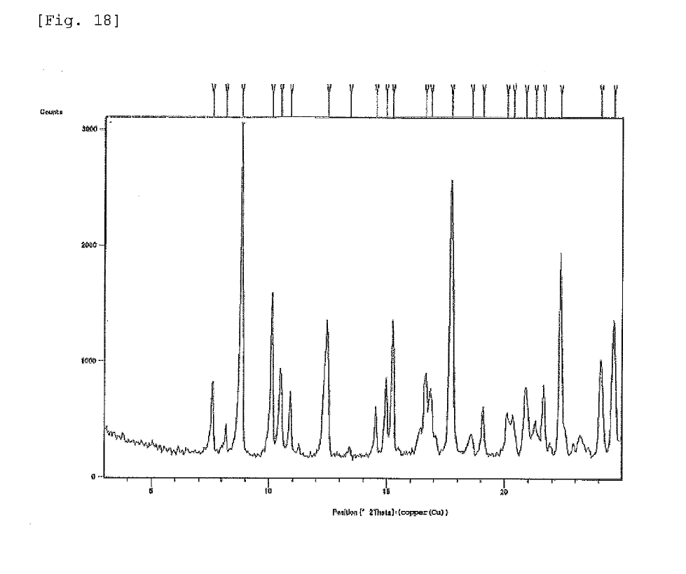

The powder X-ray diffraction pattern is shown in FIG. 18.

### Example 2-9

Compound B (50 mg) obtained in Example 2 was suspended in 3-methyl-1-butanol (0.050 ml), Form Ib crystal of compound B obtained in Example 2-2 was inoculated, and the mixture was stirred at room temperature for 7 days. The precipitated solid was collected by filtration, and dried under reduced pressure at room temperature to give compound B as Form IXb crystal (17 mg).

Form IXb crystal was subjected to powder X-ray diffraction analysis under conditions similar to those in Example 1-1.

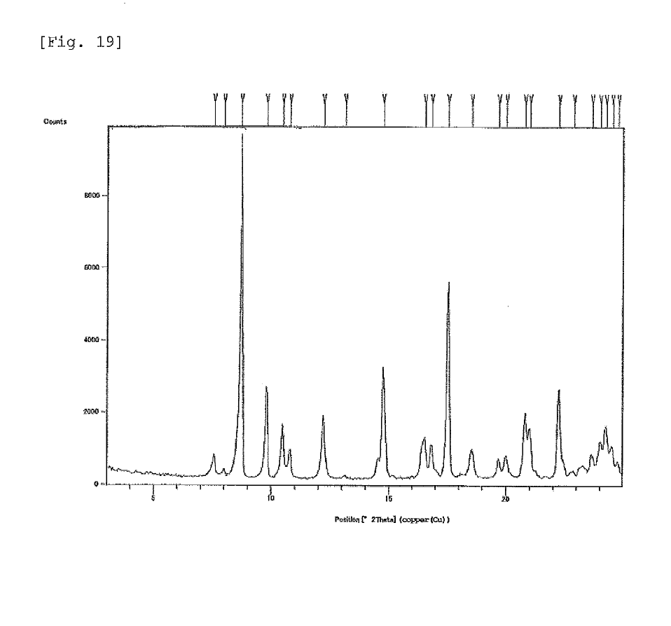

The powder X-ray diffraction pattern is shown in FIG. 19.

### Example 2-10

Form Ib crystal (50 mg) of Compound B obtained in Example 2-2 was suspended in heptane (0.20 ml), and the suspension was stirred at room temperature for 7 days. The precipitated solid was collected by filtration, and dried under reduced pressure at room temperature to give compound B as Form Xb crystal (24 mg).

Form Xb crystal was subjected to powder X-ray diffraction analysis under conditions similar to those in Example 1-1.

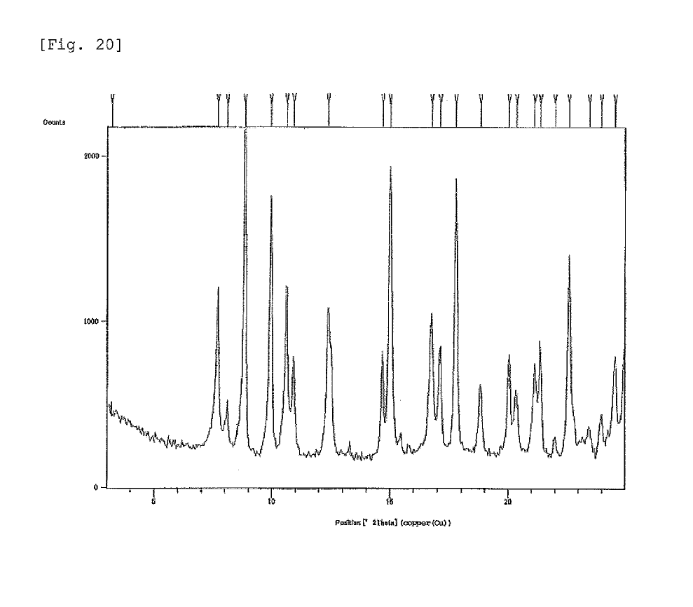

The powder X-ray diffraction pattern is shown in FIG. 20.

To obtain crystal of Compound B (including solvate), about 300 crystallization conditions including the above-mentioned Example 2-1 to Example 2-10 were tested. As a result, Form IVb crystal (2 hydrate) alone could be confirmed as an organic solvent-free crystal form.

As Formulation Examples of the present invention, the following preparations can be mentioned. However, the present invention is not limited by these Formulation Examples.

### Formulation Example 1

### Production of Capsule

| | |
|---|---|
| 1) crystal of Example 1-1 (compound (Ah)) | 30 mg |
| 2) crystalline cellulose | 10 mg |
| 3) lactose | 19 mg |
| 4) magnesium stearate | 1 mg |

1), 2), 3) and 4) are mixed and filled in a gelatin capsule.

### Formulation Example 2

### Production of Tablet

| | |
|---|---|
| 1) crystal of Example 1-1 (compound (Ah)) | 10 g |
| 2) lactose | 50 g |
| 3) cornstarch | 15 g |
| 4) carmellose calcium | 44 g |
| 5) magnesium stearate | 1 g |

The total amount of 1), 2), 3) and 30 g of 4) are kneaded with water, vacuum dried, and sieved. The sieved powder is a mixture of and 14 g of 4) and 1 g of 5), and the mixture is punched by a tableting machine. In this way, 1000 tablets each containing 10 mg of the crystal of Example 1-1 (compound (Ah)) per tablet are obtained

### Formulation Example 3

### Production of Capsule

| | |
|---|---|
| 1) crystal of Example 2-1 (compound (Bh)) | 30 mg |
| 2) crystalline cellulose | 10 mg |
| 3) lactose | 19 mg |
| 4) magnesium stearate | 1 mg |

1), 2), 3) and 4) are mixed and filled in a gelatin capsule.

### Formulation Example 4

### Production of Tablet

| | |
|---|---|
| 1) crystal of Example 2-1 (compound (Bh)) | 10 g |
| 2) lactose | 50 g |
| 3) cornstarch | 15 g |
| 4) carmellose calcium | 44 g |
| 5) magnesium stearate | 1 g |

The total amount of 1), 2), 3) and 30 g of 4) are kneaded with water, vacuum dried, and sieved. The sieved powder is a mixture of and 14 g of 4) and 1 g of 5), and the mixture is punched by a tableting machine. In this way, 1000 tablets each containing 10 mg of the crystal of Example 2-1 (compound (Bh)) per tablet are obtained.

### Experimental Example 1

### Inhibitory Action of PDHK Activity In Vitro

The inhibitory action of PDHK activity was assessed indirectly by measuring the residual PDH activity after kinase reaction in the presence of a test compound.

### (Inhibitory Action of PDHK1 Activity)

In the case of human PDHK1 (hPDHK1, Genbank Accession No. L42450.1), a 1.3 kbp fragment encoding this protein was isolated from human liver cDNA by polymerase chain reaction (PCR). Modified hPDHK1 cDNA wherein FLAG-Tag sequence was added to the N terminus was prepared by PCR and cloned into a vector (pET17b-Novagen). The recombinant construct was transformed into Escherichia coli (DH5α-TOYOB0). The recombinant clones were identified, and plasmid DNA was isolated and subjected to the DNA sequence analysis. One clone which had the expected nucleic acid sequence was selected for expression work.

For expression of hPDHK1 activity, Escherichia coli strain BL21(DE3) cells (Novagen) were transformed with the pET17b vector containing modified hPDHK1 cDNA. The Escherichia coli were grown to an optical density 0.6 (600 nmol/L) at 30° C. Protein expression was induced by the addition of 500 μmol/L isopropyl-β-thiogalactopyranoside. The Escherichia coli were cultured at 30° C. for 5 hr and harvested by centrifugation. Resuspension of the Escherichia coli paste was disrupted by a microfluidizer. FLAG-Tagged protein was purified using FLAG affinity gel (Sigma).

The gel was washed with 20 mmol/L N-(2-hydroxyethyl)piperazine-N′-2-ethanesulfonic acid-sodium hydroxide (HEPES-NaOH), 500 mmol/L sodium chloride, 1% ethylene glycol, and 0.1% polyoxyethylene-polyoxypropylene block copolymer (Pluronic F-68, pH 8.0), and the binding protein was eluted with 20 mmol/L HEPES-NaOH, 100 μg/mL FLAG peptide, 500 mmol/L sodium chloride, 1% ethylene glycol, and 0.1% Pluronic F-68 (pH 8.0).

The eluted fractions containing FLAG-Tagged protein were pooled, dialyzed against 20 mmol/L HEPES-NaOH, 150 mmol/L sodium chloride, 0.5 mmol/L ethylenediamine tetraacetic acid (EDTA), 1% ethylene glycol, and 0.1% Pluronic F-68 (pH 8.0), and preserved at −80° C. Upon the assay, the hPDHK1 enzyme concentration was set at a minimum concentration giving over 90% inhibition of PDH activity.

0.05 U/mL PDH (porcine heart PDH complex, Sigma P7032) and 1.0 μg/mL hPDHK1 were mixed in a buffer (50 mmol/L 3-morpholinopropane sulfonic acid (pH 7.0), 20 mmol/L dipotassium hydrogen phosphate, 60 mmol/L potassium chloride, 2 mmol/L magnesium chloride, 0.4 mmol/L EDTA, 0.2% Pluronic F-68, 2 mmol/L dithiothreitol), and the mixture was incubated at 4° C. overnight to obtain a PDH/hPDHK1 complex.

The test compounds were diluted with dimethyl sulfoxide (DMSO). The PDH/hPDHK1 complex (20 μL), test compound (1.5 μL) and 3.53 pmol/L ATP (diluted with buffer, 8.5 μL) were added to a half area 96 well UV-transparent microplate (Corning 3679), and PDHK reaction was performed at room temperature for 45 min. DMSO (1.5 μL) was added to control wells instead of test compound. In order to determine maximum rate of the PDH reaction, DMSO (1.5 μL) was added to blank wells instead of test compound in absence of hPDHK1.

Then, 10 μL of substrates (5 mmol/L sodium pyruvate, 5 mmol/L Coenzyme A, 12 mmol/L NAD, 5 mmol/L thiamin pyrophosphate, diluted with buffer) were added. The mixture was incubated at room temperature for 90 min, and the residual PDH activity was measured.

The absorbance at 340 nm before and after PDH reaction was measured using a microplate reader to detect NADH produced by the PDH reaction. The hPDHK1 inhibition rate (%) of the test compound was calculated from the formula [{(PDH activity of the test compound−PDH activity of control)/PDH activity of blank−PDH activity of control)}×100]. The IC₅₀ value was calculated from the concentrations of the test compound at two points enclosing 50% inhibition of the hPDHK1 activity.

The results obtained using Form I crystal of compound (Ah) as test compounds are shown in the following Table 6.

### (Inhibitory Action of PDHK2 Activity)

In the case of human PDHK2 (hPDHK2, Genbank Accession No. NM_{—}002611), modified hPDHK2 cDNA wherein FLAG-Tag sequence was added to the N terminus of hPDHK2 cDNA clone (pReceiver-M01/PDK2-GeneCopoeia) was prepared by PCR and cloned into a vector (pET17b-Novagen). The recombinant construct was transformed into Escherichia coli (DB5α-TOYOBO). The recombinant clones were identified, and plasmid DNA was isolated and subjected to the DNA sequence analysis. One clone which had the expected nucleic acid sequence was selected for expression work.

For expression of hPDHK2 activity, Escherichia coli strain BL21(DE3) cells (Novagen) were transformed with the pET17b vector containing modified hPDHK2 cDNA. The Escherichia coli were grown to an optical density 0.6 (600 nmol/L) at 30° C. Protein expression was induced by the addition of 500 μmol/L isopropyl-β-thiogalactopyranoside. The Escherichia coli were cultured at 30° C. for 5 hr and harvested by centrifugation. Resuspension of the Escherichia coli paste was disrupted by a microfluidizer. FLAG-Tagged protein was purified using FLAG affinity gel. The gel was washed with 20 mmol/L N-(2-hydroxyethyl)piperazine-N′-2-ethanesulfonic acid-sodium hydroxide (HEPES-NaOH), 500 mmol/L sodium chloride, 1% ethylene glycol, and 0.1% Pluronic F-68 (pH 8.0), and the binding protein was eluted with 20 mmol/L HEPES-NaOH, 100 μg/mL FLAG peptide, 500 mmol/L sodium chloride, 1% ethylene glycol, and 0.1% Pluronic F-68 (pH 8.0). The eluted fractions containing FLAG-Tagged protein were pooled, dialyzed against 20 mmol/L HEPES-NaOH, 150 mmol/L sodium chloride, 0.5 mmol/L ethylenediamine tetraacetic acid (EDTA), 1% ethylene glycol, and 0.1% Pluronic F-68 (pH 8.0), and preserved at −80° C. Upon the assay, the hPDHK2 enzyme concentration was set to a minimum concentration inhibiting PDH.

0.05 U/mL PDH and 0.8 μg/mL hPDHK2 were mixed in a buffer (50 mmol/L 3-morpholinopropanesulfonic acid (pH 7.0), 20 mmol/L dipotassium hydrogen phosphate, 60 mmol/L potassium chloride, 2 mmol/L magnesium chloride, 0.4 mmol/L EDTA, and 0.2% Pluronic F-68, 2 mmol/L dithiothreitol), and the mixture was incubated at 4° C. overnight to obtain a PDH/hPDHK2 complex. The test compounds were diluted with DMSO. The PDH/hPDHK2 complex (20 μL), test compound (1.5 μL) and 3.53 pmol/L ATP (diluted with buffer, 8.5 μL) were added to a half area 96 well UV-transparent microplate, and PDHK reaction was performed at room temperature for 45 min. DMSO (1.5 μL) was added to control wells instead of the test compound. In order to determine maximum rate of the PDH reaction, DMSO (1.5 μL) was added to blank wells instead of the test compound in absence of hPDHK2. Then, 10 μL of substrate (5 mmol/L sodium pyruvate, 5 mmol/L Coenzyme A, 12 mmol/L NAD, and 5 mmol/L thiamine pyrophosphate, diluted with buffer) were added. The mixture was incubated at room temperature for 90 min, and the residual PDH activity was measured. The absorbance at 340 nm before and after PDH reaction was measured using a microplate reader to detect NADH produced by the PDH reaction. The hPDHK2 inhibition rate (%) of the test compound was calculated from the formula [{(PDH activity of test compound−PDH activity of control)/PDH activity of blank−PDH activity of control)}×100]. The IC₅₀ value was calculated from the concentrations of the test compound at two points enclosing 50% inhibition of the hPDHK2 activity.

The results obtained using Form I crystal of compound (Ah) and Form IVb crystal of compound (Bh) as test compounds are shown in the following Table 6.

TABLE 6

| | hPDHK1 inhibitory | hPDHK2 inhibitory |
|---|---|---|
| test compound | activity IC₅₀ (μmol/L) | activity IC₅₀ (μmol/L) |
| Form I crystal | 0.026 | 0.014 |
| of compound (Ah) | | |
| (Example 1-1) | | |
| Form IVb crystal | not measured | 0.020 |
| of compound (Bh) | | |
| (Example 2-1) | | |

### INDUSTRIAL APPLICABILITY

Since the compound of the present invention (Ah) or a crystal thereof, or compound (Bh) or a crystal thereof, shows a PDHK inhibitory action, and is superior in stability, it is useful as a medicament for the prophylaxis or treatment of diabetes (type 1 diabetes, type 2 diabetes etc.), insulin resistance syndrome, metabolic syndrome, hyperglycemia, hyperlactacidemia, diabetic complications (diabetic neuropathy, diabetic retinopathy, diabetic nephropathy, cataract etc.), cardiac failure (acute cardiac failure, chronic cardiac failure), cardiomyopathy, myocardial ischemia, myocardial infarction, angina pectoris, dyslipidemia, atherosclerosis, peripheral arterial disease, intermittent claudication, chronic obstructive pulmonary disease, brain ischemia, cerebral apoplexy, mitochondrial disease, mitochondrial encephalomyopathy, cancer, pulmonary hypertension and Alzheimer disease.

1. A compound represented by the formula (J):

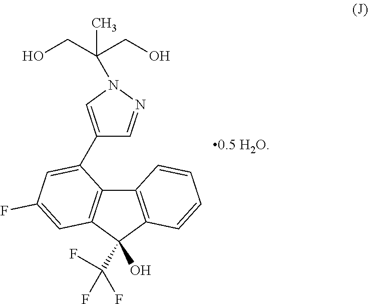 [SMILES: CC(CO)(CO)n1cc(-c2cc(F)cc3c2-c2ccccc2[C@]3(O)C(F)(F)F)cn1]

2. A crystal of the compound according to claim 1.

3. A crystal of the compound according to claim 1, having peaks at diffraction angles 2θ(°) of 6.9±0.2, 10.2±0.2, 15.5±0.2, 15.8±0.2 and 16.6±0.2 in powder X-ray diffraction.

4. A pharmaceutical composition comprising the compound according to any one of claims 1 to 3 or a crystal thereof, and a pharmaceutically acceptable carrier.

5. A method for the prophylaxis or treatment of a disease selected from the group consisting of diabetes, insulin resistance syndrome, metabolic syndrome, hyperglycemia, hyperlactacidemia, diabetic complications, cardiac failure, cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension in a mammal, comprising administering a pharmaceutically effective amount of the compound according to claim 1 or the crystal according to claim 2 or 3 to the mammal.

6. A compound represented by the formula (Q):

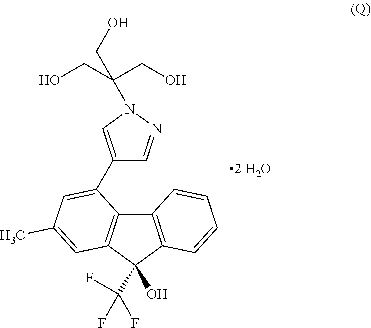 [SMILES: Cc1cc(-c2cnn(C(CO)(CO)CO)c2)c2c(c1)[C@@](O)(C(F)(F)F)c1ccccc1-2]

7. A crystal of the compound according to claim 6.

8. A crystal of the compound according to claim 6, having peaks at diffraction angles 2θ(°) of 11.8±0.2, 13.2±0.2, 14.3±0.2, 16.6±0.2 and 19.8±0.2 in powder X-ray diffraction.

9. A pharmaceutical composition comprising the compound according to any one of claims 6 to 8 or a crystal thereof, and a pharmaceutically acceptable carrier.

10. A method for the prophylaxis or treatment of a disease selected from the group consisting of diabetes, insulin resistance syndrome, metabolic syndrome, hyperglycemia, hyperlactacidemia, diabetic complications, cardiac failure, cardiomyopathy, myocardial ischemia, myocardial infarction, angina pectoris, dyslipidemia, atherosclerosis, peripheral arterial disease, intermittent claudication, chronic obstructive pulmonary diseases, brain ischemia, cerebral apoplexy, mitochondrial disease, mitochondrial encephalomyopathy, cancer and pulmonary hypertension in a mammal, comprising administering a pharmaceutically effective amount of the compound according to claim 6 or the crystal according to claim 7 or 8 to the mammal.
